(19) United States
(12) Patent Application Publication
(10) Pub. No.: US 2022/0211808 A1
(43) Pub. Date: Jul. 7, 2022

(54) HAPTOGLOBIN FOR USE IN TREATING AN ADVERSE SECONDARY NEUROLOGICAL OUTCOME FOLLOWING A HAEMORRHAGIC STROKE

(71) Applicants: UNIVERSITAET ZUERICH, Zuerich (CH); CSL BEHRING AG, Bern (CH)

(72) Inventors: Michael HUGELSHOFER, Eglisau (CH); Christian SCHAER, Zuerich (CH); Dominik SCHAER, Zuerich (CH)

(21) Appl. No.: 17/595,390

(22) PCT Filed: May 15, 2020
(86) PCT No.: PCT/EP2020/063732

§ 371 Date: Nov. 16, 2021

Related U.S. Application Data
(60) Provisional application No. 62/878,062, filed on Jul. 24, 2019.
(60) Provisional application No. 62/849,249, filed on May 17, 2019.

(51) Int. Cl. A61K 38/17; A61K 9/00; A61K 33/26; A61P 9/10

(52) U.S. Cl. CPC A61K 38/1722; A61P 9/10; A61K 33/26; A61K 9/0019

(57) ABSTRACT

The present invention relates generally to methods for treating or preventing an adverse secondary neurological outcome in a subject following a haemorrhagic stroke accompanied by extravascular erythrolysis and release of cell-free haemoglobin (Hb) into a cerebral spinal fluid (CSF) comprising exposing the CSF of a subject in need thereof to a therapeutically effective amount of haptoglobin (Hp) and for a period of time sufficient to allow the Hp, or the functional analogue thereof, to form a complex with, and thereby neutralise, the cell-free Hb. Aspects of the invention further relate to compositions and kits of an artificial CSF comprising Hp.

### TECHNICAL FIELD

The present invention relates generally to methods and compositions for treating and/or preventing an adverse secondary neurological outcome in a subject following a haemorrhagic stroke into a cerebral spinal fluid (CSF) compartment, in particular following subarachnoid hemorrhage (SAH).

### BACKGROUND

All references, including any patents or patent application, cited in this specification are hereby incorporated by reference to enable full understanding of the invention. Nevertheless, such references are not to be read as constituting an admission that any of these documents forms part of the common general knowledge in the art, in Australia or in any other country.

Haemorrhagic stroke involves the rupture of a blood vessel in or on the surface of the brain with bleeding into the surrounding tissue. Examples of haemorrhagic stroke include i) intracerebral haemorrhage (herein referred to as ICH) which involves a blood vessel in the brain bursting; ii) intraventricular haemorrhage (herein referred to as IVH) which is bleeding into the brains ventricular system; and iii) subarachnoid haemorrhage (herein referred to as SAH) which involves bleeding in the space between the brain and the tissue covering the brain known as the subarachnoid space. Most often SAH is caused by a burst aneurysm (herein referred to as aSAH). Other causes of SAH include head injury, bleeding disorders and the use of blood thinners.

Approximately 30% of IVH are primarily confined to the ventricular system of the subject and typically caused by intraventricular trauma, aneurysm, vascular malformations, or tumors, particularly of the choroid plexus. The remaining 70% are secondary in nature, resulting from an expansion of an existing haemorrhage whether intraparenchymal or SAH. IVH has been found to occur in 35% of moderate to severe traumatic brain injuries. Thus IVH usually does not occur without extensive associated damage.

Aneurysmal subarachnoid hemorrhage (aSAH) is the most common cause of SAH and is associated with the highest rates of mortality and long-term neurological disabilities^{1,2}. Despite advances in aneurysm repair and neurointensive care, the median in-hospital case fatality rate in Europe is 44.4% and 32.2% in the United States³. 35% of the survivors report a poor overall quality of life 1 year after the bleeding event with 83-94% not able to return to work⁴⁻⁶. The estimated incidence of aSAH from a ruptured intracranial aneurysm in the U.S. is 1 case per 10,000 persons, yielding approximately 27,000 new cases each year. Additionally, aSAH is more common in women than in men (2:1); the peak incidence is in persons 55 to 60 years old.

Despite the improvement in the management of haemorrhagic stroke patients and the reduction in fatalities in the last decades, disability and mortality remain high in this population. Brain injury can occur immediately and in the first days after SAH. This early brain injury can be due to physical effects on the brain such as increased intracranial pressure, herniations, intracerebral, intraventricular hemorrhage, and hydrocephalus. Subsequent adverse secondary neurological outcomes arise, including angiographic cerebral vasospasm (ACV), which, in more severe cases, can lead to delayed cerebral ischemia (DCI) and cerebral infarction. Adverse secondary neurological outcomes with the occurrence of delayed ischemic neurological deficits (DIND) will typically occur between day 4 and 14 after the initial haemorrhage or bleed and, is a major contributor to disability and mortality in these patients. DIND is a distinctive syndrome of cerebral ischaemia. Increased headache, meningism and body temperature are typically followed by a fluctuating decline in consciousness and appearance of focal neurological symptoms. A clinical DIND can be defined by either a delayed decrease of consciousness by at least two Glasgow Coma scale (GCS) levels and/or a new focal neurological deficit. Serial CT scans can be performed post-operatively, at the time of clinical deterioration and after the monitoring period to screen for delayed infarcts. This can be complemented by MRI in selected cases. The pathogenesis of DIND remains only partially understood, but is widely accepted to be multifactorial⁷. For instance, neuroinflammation plays a critical role in injury expansion and brain damage. Red blood cell breakdown products can lead to the release of inflammatory cytokines that trigger vasospasm and tissue injury⁸. Peripheral immune cells are both recruited and activated in damaged tissue. These cells can enter the brain parenchyma and release inflammatory cytokines⁹. Additionally, intrinsic toll-like receptors are upregulated after infarction leading to widespread neuroinflammation. Neuroinflammation has also been linked to adverse secondary outcomes that occur after SAH¹⁰. Vessels undergoing cerebral vasospasm (CV) have increased leukocyte adhesion capacity contributing to delayed neurologic deterioration¹⁵.

Also potentially causing brain damage are inflammation, cerebral microthrombosis, cortical spreading ischemia, blood-brain barrier breakdown, and delayed cerebral ischemia (DCI). Unfortunately, no pharmacologic treatment directed at these processes has yet shown efficacy in haemorrhagic strokes. Enteral nimodipine and the endovascular treatment of the culprit aneurysm in aSAH, remain the only treatment options supported by evidence from randomized clinical trials to improve patients' outcome. Hence, there is an urgent and unmet need for specific therapies to treat and/or prevent an adverse secondary neurological outcome in patients following a haemorrhagic stroke.

### SUMMARY OF THE INVENTION

In an aspect of the present invention, there is provided a method of treating or preventing an adverse secondary neurological outcome in a subject following a haemorrhagic stroke accompanied by extravascular erythrolysis and release of cell-free haemoglobin (Hb) into a cerebral spinal fluid (CSF), the method comprising exposing the CSF of a subject in need thereof to a therapeutically effective amount of haptoglobin (Hp), for a period of time sufficient to allow the Hp to form a complex with, and thereby neutralise, the cell-free Hb.

In another aspect disclosed herein, there is provided a pharmaceutical composition for treating or preventing an adverse secondary neurological outcome in a subject following an intraventricular haemorrhage in accordance with the method described herein, the composition comprising a therapeutically effective amount of haptoglobin (Hp) and a pharmaceutically acceptable carrier.

In another aspect disclosed herein, there is provided a pharmaceutical composition for use in treating or preventing an adverse secondary neurological outcome in a subject following an intraventricular haemorrhage in accordance with the method described herein, the composition comprising a therapeutically effective amount of haptoglobin (Hp) and a pharmaceutically acceptable carrier.

In another aspect disclosed herein, there is provided use of a therapeutically effective amount of haptoglobin (Hp) in the manufacture of a medicament for treating or preventing an adverse secondary neurological outcome in a subject following an intraventricular haemorrhage in accordance with the method described herein.

In another aspect disclosed herein, there is provided a kit comprising the artificial CSF as described herein or the composition as described herein.

### BRIEF DESCRIPTION OF THE FIGURES

FIG. 1 is an illustrative example of porcine basilar artery isolation from freshly slaughtered pigs through a transclival approach. 5-6 vascular rings per animal (length of 2 mm) were prepared under a dissection microscope while avoiding extensive mechanical vessel manipulation.

FIG. 2 shows a scheme and intraoperative photography of an experimental setup with an external ventricular drain (EVD) entering into the frontal horn of the left lateral ventricle, implanted neuromonitoring probe in the right frontal white matter and a suboccipital spinal needle.

FIG. 3 shows the semi-automated quantification of vessel diameters from Digital subtraction angiography (DSA). The left figure and the inlay on the upper right show an exemplary selection of linear regions of interest (ROIs) in the anterior cerebral artery (ACA), middle cerebral artery (MCA), cisternal part of internal carotid artery (ICA) and basilar artery (BA). These were set automatically in the straight segment of the ACA, cICA and BA with an interval of 0.5 mm (5 in number) and manually in the curvilinear segment of the MCA (3 in number). For all ROIs the vessel diameter were calculated from the intensity profile of the cross section (middle right) as described previously by Fischer et al. 2010 (bottom right) and averaged over all ROIs of the respective vessel.

FIG. 4 shows the effect of cell-free Hb in vascular function experiments with isolated porcine basilar arteries. A, After addition of MAHMA NONOate (60 nM) to the buffer, a NO-mediated vasodilatory response of the vessel segments from a porcine basilar can be observed (grey trace). In buffer containing 10 μM oxyHb (black trace) we observed no vasodilatory response after addition of MAHMA NONOate. The traces show the mean±SD of 15 and 14 vessels for buffer and buffer+Hb, respectively. B, After addition of equimolar concentration of Hp to a buffer containing 10 μM Hb, the vasodilatory response to MAHMA NONOate is restored. The traces show the mean±SD of 16 vessels per group. In this experiment the vascular response was normalized relative to the tension after passive stretching to the optimal IC1/IC100 ratio (=100%) and the level of tonic contraction after addition of PF2α (=0%).

FIG. 5 shows an image and absorption spectra of a (centrifuged) erythrolytic patient CSF sample before (dark) and after selective removal of Hb (bright) by an Hp-column. Hb removal restored the vasodilatory response to MAHMA NONOate administration as indicated by the transient decrease of the tension records of porcine basilar artery segments that were immersed in CSF (grey). The dilatory NO-signal remains uncoupled in the same CSF sample before Hb-depletion (black).

FIG. 6 shows dilatory NO-signal coupling in porcine basilar arteries immersed in CSF from patients with aSAH (n=9) after administration of MAHMA-NONOate to the CSF. The arteries were sequentially probed in pre-haemolytic CSF (left panel), haemolytic CSF (middle panel) and after the addition of Hp to the haemolytic CSF (right panel).

FIG. 7 shows the circle of Willis on digital subtraction angiography, on a photograph of an anatomical specimen and on a curved multiplanar reconstruction (curved MPR) of a T1 weighted MR image. The bright (white) signal in the curved MPR represents the infused hemoprotein (Hb-Hp complex in this example), surrounding the posterior communicating artery (PCOM) and the basilar artery (BA), as indicated in the coronal view images. The dashed lines (1-3) in the curved MPR indicate the location of the coronal sections.

FIG. 8 shows representative histological images of ovine brain sections through the lateral ventricles after injection of TCO-labeled Hb (A) and TCO-labelled Hb:Hp (B), stained for nuclei (“Nuclei”) and the labeled compound (“TCO-Hb” or “TCO-HbHp”). A, Hb penetrates from the ventricular system through the ependymal barrier into the brain interstitial space (bright areas in “TCO-Hb” images in A). B, Penetration of Hb:Hp complexes through the ependymal barrier into the brain interstitial space is drastically reduced compared to Hb alone. However, if the integrity of the ependymal barrier is disturbed (e.g. local damage after implantation of an EVD), Hb:Hp locally penetrates into the brain tissue (arrow head). Whole slide scans were produced by stitching single images obtained with a 10× magnification. The upper panels display an overlay of a nuclear stain (Hoechst) and the TCO-labeled Hb or Hb:Hp respectively (Tet-Cy5), whereas the lower panel show only the labeled protein.

FIG. 9 shows representative histological images of ovine brain sections section through the mesencephalon after injection of TCO-labeled Hb (A) and TCO-labelled Hb:Hp (B), stained for nuclei (“Nuclei”) and the labeled compound (“TCO-Hb” or “TCO-HbHp”). A, Hb penetrates from the cranial subarachnoid CSF space through the glia limitans of the mesencephalon into the brain parenchyma (bright areas in “TCO-Hb” images in A). B, The distribution of Hb:Hp complexes is restricted to the CSF-filled perivascular spaces (Virchow-Robin-Spaces) of penetrating cortical vessels. Whole slide scans were produced by stitching single images obtained with a 10× magnification. The upper panels display an overlay of a nuclear stain and the labeled compound, whereas the lower panel show only the labeled compound.

FIG. 10 shows representative confocal images of small arteries in the periventricular area of the midbrain from a sheep after infusion of TCO-labelled Hb (A-D) or Hb:Hp complexes (E-H). The 120 μm vibratome sections were stained for vascular smooth muscle cells (aSMA), astrocyte end-feet (AQP4) and TCO-labelled Hb (tetrazine-Cy5). Black to white gradient images (Hb signal) display only the signal of the labelled hemoprotein from the corresponding image. The delocalization of cell-free Hb from the CSF into vascular structures (smooth muscle cell layer) and the brain parenchyma (astrocyte area) is blocked by Hp. Scale bars are 20 μm.

FIG. 11 shows a comparison of illustrative DSA in lateral projection of two sheep after infusion of Hb (A) or Hb:Hp complexes (B). Segmental vasospasms of the basilar artery (arrow) were apparent 60 min after infusion of Hb, whereas no segmental vasospasms could be detected in animals infused with Hb:Hp complexes.

FIG. 12 shows illustrative baseline (A) and 60 min after Hb infusion (B) angiograms of segmental vasospasms in the middle cerebral artery in the lateral projection (arrowheads, upper panel) and of the anterior cerebral artery (arrow, lower panel) as well as of the middle cerebral artery (asterisk, lower panel) in the dorsoventral projection. The images show segmental vasospasms occurring in all major vascular territories.

FIG. 13. The upper panel (A) shows the relative change in diameter of cerebral arteries 60 minutes after infusion of Hb or Hb:Hp (ACA: anterior cerebral artery, BA: basilar artery, ICA: internal carotid artery, MCA: middle cerebral artery). Diamonds represent the mean and the 95% confidence interval (n=4 sheep per group). The lower panel shows cumulative analysis of the relative diameter changes of all analyzed arteriel regions 60 minutes after infusion of aCSF (B), Hb or Hb:Hp (C). Diamonds represent the mean and the 95% confidence interval. (ACA: anterior cerebral artery, BA: basilar artery, ICA: internal carotid artery, MCA: middle cerebral artery).

FIG. 14 shows (A) sequential SEC elution profiles of sheep CSF samples collected from the subarachnoid space after intraventricular infusion of Hb and (later) Hp, as indicated. Standard elution profiles of Hb and Hb-Hp complexes are shown on the top; and (B) DSA of the middle cerebral artery (MCA) at baseline, 45 minutes after Hb infusion and 45 minutes after Hp infusion.

FIG. 15 shows coupling of NO-mediated relaxation of porcine basilar arteries that were immersed in sheep CSF collected before or after the 60 min post-treatment angiograms. After infusion of artificial CSF (CSF-heme OpM Hb=ctrl CSF; left panel), after Hb infusion (CSF-heme 200-240 μM Hb; middle panel): after Hb-Hp infusion (CSF-heme 200-240 μM; right panel). The dotted line in the middle panel shows rescue of NO-response through ex vivo addition of equimolar Hp. Dilatory responses were induced with a single bolus of MAHMA-NONOate in all experiments.

FIG. 16 shows the classification of CSF proteins in patient samples at days 1, 4, 7, 11, and 14 after the acute bleeding. Proteins identified by LC-MSMS were analyzed by K-means clustering of the log-transformed normalized ion intensity ratios. The right panel shows a principal component analysis of the identified proteins. Cluster 1: proteins remaining unchanged (i.e. ALB). Cluster 2: proteins decreasing over time (i.e. HP, HPR). Cluster 3: proteins increasing over time (i.e. HBB, HBA, FTH, HBD, CAT, CA1, FTL). Abbreviations: ALB, albumin; HP, haptoglobin; HPR, haptoglobin related protein; HBB, Hb-beta; HBA, Hb-alpha; FTH, ferritin heavy-chain; HBD, Hb-delta; CAT, catalase; CA1, carbonic anhydrase; FTL, ferritin light-chain.

FIG. 17 shows a histomorphometric analysis of arterioles in the tela choroidea of the fourth Ventricle. 120 μm sections of sheep brain were stained for alpha-smooth muscle actin (aSMA). (A) Illustration of the anatomical situation of the studied brain sections. A researcher blinded to the treatments recorded confocal images of the vessels in the tela choroidea. (B) Based on the manual delineation of the inner and outer circumference of the aSMA positive structures with Image J software, the lumen area (Aᵢₙₙₑᵣ), and total sectional vessel area (Aₒᵤₜₑᵣ) were quantified for each vessel, and the lumen area fraction was calculated. These measurements were performed for all images by three blinded researchers, and the mean values were further analyzed. (C) Representative images of arterioles in the tela choroidea of a Hb and a Hb-haptoglobin treated sheep. (D) Plotted data and statistical analysis for all analyzed vessels (n=57). (E) Plotted data and statistical analysis for the mean lumen area per sheep (n=3). Dots represent individual animals. The diamonds represent the mean and 95% Cl. The overlap marks (horizontal lines above and below the mean-line) define statistical significant difference between groups if not overlapping (p<0.05).

FIG. 18 shows the response of isolated basilar artery segments immersed in buffer containing 10 μM oxyHb to MAHMA NONOate (60 nM) to the buffer before (left two boxplots) and after addition of either recombinant Hp 1-1 or plasma-derived Hp 2-2. No significant difference in recovery of the NO-induced vasodilatory response can be observed between Hp 1-1 and Hp 2-2. N=4 vascular segments per tested condition. In this experiment the vascular response was normalized relative to the tension after passive stretching to the optimal IC1/IC100 ratio (=100%) and the level of tonic contraction after addition of PF2α (=0%).

FIG. 19 shows the response of isolated basilar artery segments immersed in buffer containing 10 μM oxyHb to MAHMA NONOate (60 nM) to the buffer before (“Hb dips” upper pannel) and after (“Hp dips” lower pannel) addition of either equimolar concentration of plasma-derived Hp 1-1 (black dotted traces) or plasma-derived Hp 2-2 (grey traces). No qualitative difference in recovery of the vasodilatory response to NO can be seen between plasma-derived Hp 1-1 and Hp 2-2. N=6 vascular segments per tested condition. In this experiment the vascular response was normalized relative to the tension after passive stretching to the optimal IC1/IC100 ratio (=100%) and the level of tonic contraction after addition of PF2α (=0%).

### DETAILED DESCRIPTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The reference in this specification to any prior publication (or information derived from it), or to any matter which is known, is not, and should not be taken as an acknowledgment or admission or any form of suggestion that prior publication (or information derived from it) or known matter forms part of the common general knowledge in the field of endeavour to which this specification relates.

It must be noted that, as used in the subject specification, the singular forms “a”, “an” and “the” include plural aspects unless the context clearly dictates otherwise. Thus, for example, reference to “an agent” includes a single resin, as well as two or more agents; reference to “the composition” includes a single composition, as well as two or more compositions; and so forth.

In the absence of any indication to the contrary, reference made to a “%” content throughout this specification is to be taken as meaning % w/w (weight/weight). For example, a solution comprising a haptoglobin content of at least 80% of total protein is taken to mean a composition comprising a haptoglobin content of at least 80% w/w of total protein.

The present invention is predicated, at least in part, on the inventors' surprising finding that Hp can reduce or otherwise prevent cell-free Hb-mediated adverse secondary neurological outcomes, such as cerebral vasospasm, in vivo. Thus, in an aspect disclosed herein, there is provided a method of treating or preventing an adverse secondary neurological outcome in a subject following a haemorrhagic stroke accompanied by extravascular erythrolysis and release of cell-free haemoglobin (Hb) into a cerebral spinal fluid (CSF), the method comprising exposing the CSF of a subject in need thereof to a therapeutically effective amount of haptoglobin (Hp) for a period of time sufficient to allow the Hp, or the functional analogue thereof, to form a complex with, and thereby neutralise, the cell-free Hb.

### Haemorrhagic Stroke

Haemorrhagic stroke, or bleeding, into the CSF compartment, is also referred to interchangeably herein as a brain haemorrhage, a cerebral haemorrhage or an intracranial haemorrhage. It is typically characterised by a ruptured blood vessel in the brain causing localized bleeding. The location of the bleed can vary. For example, haemorrhage into the CSF compartment may result from an intraventricular haemorrhage, an intraparenchymal haemorrhage, and/or a subarachnoid haemorrhage.

Haemorrhagic stroke is made up of a range of pathologies with different natural courses, assessment, and management, as will be familiar to persons skilled in the art. It is generally categorized as primary or secondary, depending on aetiology.

In an embodiment, the haemorrhagic stroke is an intraventricular haemorrhage (IVH) or a subarachnoid haemorrhage (SAH). In an embodiment, the haemorrhagic stroke is an aneurysmal subarachnoid haemorrhage (aSAH).

Methods of diagnosing a haemorrhagic stroke, and in particular SAH, in a subject will be familiar to persons skilled in the art, illustrative examples of which include cerebral angiography, computerised tomography (CT) and spectrophotometric analysis of oxyHb and bilirubin in the subject's CSF (see, for example, Cruickshank A M., 2001, ACP Best Practice No 166, J. Clin. Path., 54(11):827-830).

It will be understood by persons skilled in the art that the haemorrhagic stroke can be a spontaneous haemorrhage (e.g., as a result of a ruptured aneurysm) or a traumatic haemorrhage (e.g., as a result of a trauma to the head). In an embodiment, the haemorrhagic stroke is a spontaneous haemorrhage, also known as a non-traumatic haemorrhage. In an embodiment, the haemorrhagic stroke is a traumatic haemorrhage.

The term “cerebrospinal fluid”, or CSF, is understood to mean the fluid within the brain ventricles and the cranial and spinal subarachnoid spaces. The brain ventricles, cranial and spinal subarachnoid spaces are collectively referred to herein as the “CSF compartment”. Thus, in an embodiment disclosed herein, the method comprises exposing the CSF compartment of the subject in need thereof to a therapeutically effective amount of Hp.

CSF is predominantly, but not exclusively, secreted by the choroid plexuses, which consist of granular meningeal protrusions into the ventricular lumen, the epithelial surface of which is continuous with the ependyma. Studies have also suggested that brain interstitial fluid, ependyma and capillaries may also play a role in CSF secretion. The CSF volume is estimated to be about 150 mL in human adults, with a typically distribution of between 125 mL in cranial and spinal subarachnoid spaces and 25 mL in the ventricles, albeit with marked variation between individuals. CSF secretion in human adults varies between 400 to 600 mL per day, with about 60-75% of CSF produced by the choroid plexuses of the lateral ventricles and the tela choroidea of the third and fourth ventricles. Choroidal secretion of CSF typically comprises two steps: (i) passive filtration of plasma from choroidal capillaries to the choroidal interstitial compartment according to a pressure gradient and (ii) active transport from the interstitial compartment to the ventricular lumen across the choroidal epithelium, involving carbonic anhydrase and membrane ion carrier proteins. CSF plays an essential role in homeostasis of cerebral interstitial fluid and the neuronal environment by regulation of the electrolyte balance, circulation of active molecules, and elimination of catabolites. CSF transports the choroidal plexus secretion products to their sites of action, thereby modulating the activity of certain regions of the brain by impregnation, while synaptic transmission produces more rapid changes of activities. The wastes of brain metabolism, peroxidation products and glycosylated proteins, accumulate with age-related decreased CSF turnover (Sakka et al., 2011, European Annals of Otorhinolaryngology, Head and Neck Diseases, 128(6):309-316).

### Adverse Secondary Neurological Outcome

Patients who survive a haemorrhagic stroke, such as SAH, are at significant risk of developing one or more adverse secondary neurological outcomes or complications. The term “adverse secondary neurological outcome”, as used herein, refers to an adverse neurological event (secondary injury to brain tissue) that follows a haemorrhagic stroke. Secondary injury after haemorrhagic stroke may be caused by a cascade of events initiated by the primary injury (e.g., mass effect and physical disruption), by the physiological response to the haematoma (e.g. inflammation), and/or by the release of blood and blood components. Adverse secondary neurological outcomes will be familiar to persons skilled in the art, illustrative examples of which include delayed ischaemic neurological deficit (DIND), delayed cerebral ischaemia (DCI), neurotoxicity, apoptosis, inflammation, nitric oxide depletion, oxidative tissue injury, cerebral vasospasm, cerebral vasoreactivity, oedema and spreading depolarisation (see, for example, Al-Tamimi et al., World Neurosurgery, 73(6):654-667 (2010); Macdonald et al., Neurocrit. Care, 13:416-424 (2010); and Macdonald et al., J. Neurosurg. 99:644-652 (2003)).

The terms “treating”, “treatment”, “treat” and the like, are used interchangeably herein to mean relieving, minimising, reducing, alleviating, ameliorating or otherwise inhibiting an adverse secondary neurological outcome, including one or more symptoms thereof, as described herein. The terms “treating”, “treatment” and the like are also used interchangeably herein to mean preventing an adverse secondary neurological outcome from occurring or delaying the onset or subsequent progression of an adverse secondary neurological outcome in a subject that may be predisposed to, or at risk of, developing an adverse secondary neurological outcome, but has not yet been diagnosed as having it. In that context, the terms “treating”, “treatment” and the like are used interchangeably with terms such as “prophylaxis”, “prophylactic” and “preventative”. It is to be understood, however, that the methods disclosed herein need not completely prevent an adverse secondary neurological outcome from occurring in the subject to be treated. It may be sufficient that the methods disclosed herein merely relieve, reduce, alleviate, ameliorate or otherwise inhibit an adverse secondary neurological outcome in the subject to the extent that there are fewer adverse secondary neurological outcomes and/or less severe adverse secondary neurological outcomes than would otherwise have been observed in the absence of treatment. Thus, the methods described herein may reduce the number and/or severity of adverse secondary neurological outcomes in the subject following haemorrhagic stroke.

It is to be understood that a reference to a subject herein does not imply that the subject has had a haemorrhagic stroke, but also includes a subject that is at risk of a haemorrhagic stroke. In an embodiment, the subject has (i.e., is experiencing) a haemorrhagic stroke, or a symptom thereof. In another embodiment, the subject has not had a haemorrhagic stroke at the time of treatment, but is at risk of a haemorrhagic stroke. As an illustrative example, the subject has an aneurysm that has not yet ruptured but is at risk of rupture. In this instance, the subject may undergo surgical intervention to minimise the risk of rupture of the aneurysm (e.g., by surgical clipping or endovascular coiling). The methods described herein may therefore suitably be prescribed to the subject as a prophylactic measure to minimise, reduce, abrogate or otherwise inhibit an adverse secondary neurological outcome should the aneurysm rupture prior to, during or subsequent to the surgical intervention. In that context, the methods described herein may be employed as a prophylactic measure prior to, during or subsequent to surgical intervention.

The extent to which the methods disclosed herein provide a subjective, qualitative and/or quantitative reduction in the number and/or severity of adverse secondary neurological outcomes following haemorrhagic stroke may be represented as a percentage reduction, for example, by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, preferably from about 40% to about 50%, preferably from about 45% to about 55%, preferably from about 50% to about 60%, preferably from about 55% to about 65%, preferably from about 60% to about 70%, preferably from about 65% to about 75%, preferably from about 70% to about 80%, preferably from about 75% to about 85%, preferably from about 80% to about 90%, preferably from about 85% to about 95%, or most preferably from about 90% to 100% when compared to the number and/or severity of adverse secondary neurological outcomes prior to exposing the CSF to the therapeutically effective amount of Hp.

Suitable methods by which a subjective, qualitative and/or quantitative reduction in the number and/or severity of adverse secondary neurological outcomes can be measured following a haemorrhagic stroke will be familiar to persons skilled in the art and will largely depend on the nature of the adverse secondary neurological outcome to be measured. Illustrative examples are described elsewhere herein.

In an embodiment, the adverse secondary neurological outcome is selected from the group consisting of delayed ischaemic neurological deficit (DIND), delayed cerebral ischaemia (DCI), neurotoxicity, inflammation, nitric oxide depletion, oxidative tissue injury, cerebral vasospasm, cerebral vasoreactivity, oedema and spreading depolarisation.

In an embodiment, the adverse secondary neurological outcome is a delayed ischaemic neurological deficit (DIND). DIND after SAH is a serious and poorly understood syndrome of cerebral ischaemia characterised by increased headache, meningism and/or body temperature, typically followed by a fluctuating decline in consciousness and appearance of focal neurological symptoms. DIND is characteristically defined as deterioration in neurological function seen at least 3 to 4 days post-haemorrhagic ictus. It is also referred to as clinical/symptomatic vasospasm or delayed cerebral ischemia (DCI). DIND remains a significant cause of morbidity and mortality in survivors of the initial haemorrhage. The reported prevalence of DIND is about 20% to 35%, although in those with a higher blood load, this may be as high as 40%. DIND has been attributed to cerebral infarcts in approximately 20% of patients and to about 13% of all death and disability after aSAH. Suitable methods of determining DIND will be familiar to persons skilled in the art, illustrative examples of which are described in Dreier et al., Brain, 2006; 129(12): 3224-3237, the contents of which are incorporated herein by reference in their entirety. In an embodiment, DIND is determined by spreading mass depolarization, as evidence, for example, by spreading negative slow voltage variations by electrocorticography. In an embodiment, DIND is associated with a delayed decrease of consciousness by at least two GCS levels and/or a new focal neurological deficit.

In an embodiment, the adverse secondary neurological outcome is a cerebral vasospasm. Cerebral vasospasm, or CV (also referred to as “angiographic cerebral vasospasm”), is one of the most common causes of focal ischaemia after a haemorrhagic stroke and can account for up to about 23% of SAH-related disability and death. CV is typically characterised by narrowing of the blood vessels caused by persistent contraction of blood vessels, in particular of the large capacitance arteries at the base of the brain (i.e., the cerebral arteries) following a haemorrhagic stroke into the subarachnoid space. The term “vasospasm” is therefore typically used with reference to angiographically determined arterial narrowing. The persistent contraction of blood vessels reduces perfusion of distal brain regions and increased cerebral vascular resistance. Left untreated, CV can ultimately lead to neurotoxicity (brain cell damage) in the form of cerebral ischaemia and infarction, primarily due to the restricted blood supply to brain tissue. CV can be detected by any suitable means known to persons skilled in the art, illustrative examples of which include digital subtraction angiography (DSA), computed tomography (CT) angiography (CTA), magnetic resonance (MR) angiography (MRA), Transcranial Doppler ultrasonography and catheter (cerebral) angiography (CA). In an embodiment, CV is detected by digital subtraction angiography (DSA). Without being bound by theory or a particular mode of application, vasospasm of the cerebral arteries will typically begin about 3 days after SAH, peak at about 7 to 8 days later and resolve by about 14 days (see, e.g., Weir et al., 51., 48:173-178 (1978)), with some degree of angiographic narrowing occurring in at least two-thirds of patients having angiography between 4 and 12 days after SAH.

The incidence of CV depends on the time interval after the SAH. As noted elsewhere herein, peak incidence typically occurs about 7-8 days after SAH (range, 3-12 days). In addition to the time after the SAH, other principal factors that affect the prevalence of vasospasm are the volume, density, temporal persistence and distribution of subarachnoid blood. Prognostic factors for CV may include the amount of subarachnoid blood on CT scan, hypertension, anatomical and systemic factors, clinical grade and whether the patient is receiving antifibrinolytics.

Symptoms of CV typically develop sub-acutely and may fluctuate and can include excess sleepiness, lethargy, stupor, hemiparesis or hemiplegia, aboulia, language disturbances, visual fields deficits, gaze impairment, and cranial nerve palsies. Although some symptoms are localized, they are generally not diagnostic of any specific pathological process. Cerebral angiography is typically employed as the gold standard for visualizing and studying cerebral arteries, although Transcranial Doppler ultrasonography can also be used.

As noted elsewhere herein, the present inventors have surprisingly found that Hp can reduce or otherwise prevent cell-free Hb-mediated CV in vivo. It is to be understood that the extent to which the methods disclosed herein reduce or otherwise prevent Hb-mediated CV may depend on several factors, such as the degree of vasoconstriction (vessel narrowing) that is induced by cell-free Hb following a haemorrhagic stroke, the concentration of cell-free Hb in the subject's CSF following a haemorrhagic stroke, the time period to which the CSF is exposed to the Hp and the presence or absence of any persistence bleeding. Means of assessing whether the methods disclosed herein have reduced or otherwise prevented CV in the subject will be familiar to persons skilled in the art, illustrative examples of which include digital subtraction angiography (DSA), computed tomography (CT) angiography (CTA), magnetic resonance (MR) angiography (MRA) and catheter angiography (CA).

In an embodiment, exposing the CSF of a subject in need thereof to the Hp, as described herein, restores the average diameter of the lumen of a constricted cerebral blood vessel by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, or more preferably from about 40% to about 50% following a 60 minute period of exposure, as determined by DSA.

In an embodiment, exposing the CSF of a subject in need thereof to the Hp, as described herein, restores the average diameter of the lumen of a constricted anterior cerebral artery by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, or more preferably from about 40% to about 50% following a 60 minute period of exposure, as determined by DSA.

In an embodiment, exposing the CSF of a subject in need thereof to the Hp, as described herein, restores the average diameter of the lumen of a constricted internal carotid artery by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, or more preferably from about 40% to about 50% following a 60 minute period of exposure, as determined by DSA.

In an embodiment, exposing the CSF of a subject in need thereof to the Hp, as described herein, restores the average diameter of the lumen of a constricted medial cerebral artery by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, or more preferably from about 40% to about 50% following a 60 minute period of exposure, as determined by DSA.

In an embodiment, exposing the CSF of a subject in need thereof to the Hp, as described herein, restores the average diameter of the lumen of a constricted basilar artery by at least 10%, preferably from about 10% to ab out 20%, preferably from about 15% to about 25%, preferably from about 20% to about 30%, preferably from about 25% to about 35%, preferably from about 30% to about 40%, preferably from about 35% to about 45%, or more preferably from about 40% to about 50% following a 60 minute period of exposure, as determined by DSA.

In an embodiment, exposing the CSF of a subject in need thereof to the Hp, as described herein, increases the average lumen area of a constricted small parenchymal vessel (e.g., a cerebral arteriole) by at least 10%, preferably by at least 20%, preferably by at least 25%, preferably by at least 30%, preferably by at least 35%, preferably by at least 40%, preferably by at least 45%, preferably by at least 55%, preferably by at least 60%, preferably by at least 65%, preferably by at least 70%, preferably by at least 75%, or more preferably by at least 80% following a 60 minute period of exposure, as determined by in vivo perfusion imaging (e.g. MRI perfusion, CT perfusion). In an embodiment, the small parenchymal vessel is a cerebral arteriole. In an embodiment, exposing the CSF of a subject in need thereof to the Hp, as described herein, restores the cerebral microperfusion by prevention of small parenchymal vessel constriction by at least 10%, preferably by at least 20%, preferably by at least 25%, preferably by at least 30%, preferably by at least 35%, preferably by at least 40%, preferably by at least 45%, preferably by at least 55%, preferably by at least 60%, preferably by at least 65%, preferably by at least 70%, preferably by at least 75%, or more preferably by at least 80% following a 60 minute period of exposure, as determined by in vivo perfusion imaging (e.g. MRI perfusion, CT perfusion). In an embodiment, the small parenchymal vessel is a cerebral arteriole.

In an embodiment, the adverse secondary neurological outcome is delayed cerebral ischaemia (DCI). DCI typically occurs in around a third of patients with aSAH and causes death or permanent disability in half of these patients (Dorsch and King, Journal of Clinical Neuroscience, 1:19-26 (1994)). DCI is often characterised as delayed neurological deterioration resulting from tissue ischemia and is usually associated with the occurrence of focal neurological impairment such as hemiparesis, aphasia, apraxia, hemianopia, or neglect, and/or a decrease in the Glasgow coma scale (either the total score or one of its individual components [eye, motor on either side, verbal]) (see, e.g., Frontera et al., Stroke, 40:1963-1968 (2009); Kassell et al., J. Neurosurg., 73:18-36 (1990); and Vergouwen et al., Stroke, 41:e47-e52 (2010)). This may last for at least one hour, is not apparent immediately after aneurysm occlusion and cannot be attributed to other causes by means of clinical assessment, CT or MRI scanning of the brain. DCI and development of delayed cerebral infarction are among the most important causes of poor outcome after SAH.

Cerebral infarction may also be a consequence of DCI. For instance, infarction due to DCI is typically defined as the presence of an area of brain cell death resulting from insufficiency of arterial or venous blood supply to the brain. It can be detected by CT or MRI scan of the brain within about 6 weeks after SAH, or on the latest CT or MRI scan made before death within about 6 weeks, or proven at autopsy, not present on the CT or MRI scan between about 24 and 48 hours after early aneurysm occlusion, and not attributable to other causes such as surgical clipping or endovascular treatment. Hypodensities on CT imaging resulting from ventricular catheter or intraparenchymal hematoma generally are not regarded as evidence of cerebral infarction from DCI.

As reported by Vergouwen et al. (Stroke. 2010; 41:2391-2395), uniform definitions of “clinical deterioration caused by delayed cerebral ischemia” and “cerebral infarction” should capture the most relevant elements in terms of morphological and clinical characteristics, without assumptions about its pathogenesis. Because cerebral infarction on CT/MRI is strongly correlated with functional outcome 3 months after SAH, and given its expected high interobserver agreement rate, its ability to detect DCI in sedated and comatose patients, and its objective quantification of the consequences of DCI, cerebral infarction on neuroimaging might be a better outcome measure than clinical deterioration caused by DCI alone. Although previous definitions of DCI often combined clinical features of DCI with either angiography/transcranial Doppler findings or cerebral infarction on neuroimaging or autopsy, the authors suggest that these should be separately reported. They also suggest that clinical deterioration caused by DCI should be not more than a secondary measure of outcome, because of suspected lower interobserver agreement rates. According to Vergouwen et al., the proposed definition of clinical deterioration caused by DCI is: “The occurrence of focal neurological impairment (such as hemiparesis, aphasia, apraxia, hemianopia, or neglect), or a decrease of at least 2 points on the Glasgow Coma Scale (either on the total score or on one of its individual components [eye, motor on either side, verbal]). This should last for at least 1 hour, is not apparent immediately after aneurysm occlusion, and cannot be attributed to other causes by means of clinical assessment, CT or MRI scanning of the brain, and appropriate laboratory studies.”

Adverse secondary neurological outcomes following haemorrhagic stroke, including SAH, have also been shown to be associated with inflammation, including immune cell activation and/or infiltration into the CSF compartment and the release of inflammatory cytokines. As discussed by Miller et al. (Biomed Res Int. 2014; 2014: 384342), studies have shown that inflammation is a direct mediator of neurological injury after SAH and a causative factor of post-SAH vasospasm. Key inflammatory molecules implicated in the pathophysiology of SAH will be familiar to persons skilled in the art, illustrative examples of which include selectins (L-selectin and P-selectin), integrins (e.g., lymphocyte function-associated antigen 1 (LFA-1) and Mac-1 integrin (CD11b/CD18)), TNFα, monocyte chemoattractant protein 1 (MCP-1), Intercellular Adhesion Molecule 1 (ICAM-1), pro-inflammatory interleukins (e.g., IL-1, IL-6, IL-1B, IL-8) and endothelin 1 (ET-1). In an embodiment disclosed herein, an adverse secondary neurological outcome is associated with differential expression of one or more inflammatory markers selected from the group consisting of a selectin (e.g., L-selectin and P-selectin), an integrin (e.g., lymphocyte function-associated antigen 1 (LFA-1) and Mac-1 integrin (CD11b/CD18)), TNFα, monocyte chemoattractant protein 1 (MCP-1), Intercellular Adhesion Molecule 1 (ICAM-1), a pro-inflammatory interleukin and endothelin 1 (ET-1). In an embodiment, the pro-inflammatory interleukin is selected from the group consisting of IL-1, IL-6, IL-1B and IL-8.

Nissen et al. have previously shown that the serum concentration of P-selectin in patients with DIND is significantly higher when compared to patients without DIND (J Neurol Neurosurg Psychiatry 2001; 71:329-333). The authors also showed that the serum concentration of L-selectin in patients with DIND is significantly lower when compared to patients without DIND. Thus, in an embodiment, the extent to which the methods described herein reduce the number and/or severity of adverse secondary neurological outcomes following haemorrhagic stroke is determined by a reduction in the concentration of P-selectin in the serum or CSF of the subject, for example, by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, preferably from about 40% to about 50%, preferably from about 45% to about 55%, preferably from about 50% to about 60%, preferably from about 55% to about 65%, preferably from about 60% to about 70%, preferably from about 65% to about 75%, preferably from about 70% to about 80%, preferably from about 75% to about 85%, preferably from about 80% to about 90%, preferably from about 85% to about 95%, or most preferably from about 90% to 100% when compared to the concentration of P-selectin in the subject prior to treatment. In another embodiment, the extent to which the methods described herein reduce the number and/or severity of adverse secondary neurological outcomes following haemorrhagic stroke is determined by an increase in the concentration of L-selectin in the serum or CSF of the subject, for example, by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, preferably from about 40% to about 50%, preferably from about 45% to about 55%, preferably from about 50% to about 60%, preferably from about 55% to about 65%, preferably from about 60% to about 70%, preferably from about 65% to about 75%, preferably from about 70% to about 80%, preferably from about 75% to about 85%, preferably from about 80% to about 90%, preferably from about 85% to about 95%, or most preferably from about 90% to 100% when compared to the concentration of L-selectin in the subject prior to treatment. Methods by which the concentration of P-selectin and L-selectin can be measured will be familiar to persons skilled in the art, illustrative examples of which are described in Nissen et al. (J Neurol Neurosurg Psychiatry 2001; 71:329-333), the contents of which are incorporated herein by reference in their entirety.

The level of proinflammatory cytokines IL-1B, IL-6, IL-8, TNFα, and MCP-1, as well as endothelin-1, have also been shown to be elevated in patients following SAH (Miller et al. Biomed Res Int. 2014; 2014: 384342). Thus, in an embodiment disclosed herein, the extent to which the methods described herein reduce the number and/or severity of adverse secondary neurological outcomes following haemorrhagic stroke is determined by a reduction in the concentration of a proinflammatory cytokine in the serum or CSF of the subject, for example, by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, preferably from about 40% to about 50%, preferably from about 45% to about 55%, preferably from about 50% to about 60%, preferably from about 55% to about 65%, preferably from about 60% to about 70%, preferably from about 65% to about 75%, preferably from about 70% to about 80%, preferably from about 75% to about 85%, preferably from about 80% to about 90%, preferably from about 85% to about 95%, or most preferably from about 90% to 100% when compared to the concentration of the proinflammatory cytokine in the subject prior to treatment, wherein the proinflammatory molecule is selected from the group consisting of IL-1B, IL-6, IL-8, TNFα, MCP-1 and endothelin-1. Methods by which the concentration of inflammatory mediators, as described herein, can be measured will be familiar to persons skilled in the art.

Nitric oxide (NO) depletion in CSF has also been shown to contribute to the pathogenesis of adverse secondary neurological outcomes following haemorrhagic stroke (see Pluta et al. JAMA. 2005; 293(12):1477-1484). NO levels are decreased in CSF after SAH due to (1) toxicity of oxyhemoglobin to neurons containing neuronal nitric oxide synthase (NOS) in the adventitia of the artery; (2) endogenous inhibition of endothelial NOS; and (3) scavenging of nitric oxide by oxyhemoglobin released from the subarachnoid clot. Thus, in an embodiment disclosed herein, the extent to which the methods described herein reduce the number and/or severity of adverse secondary neurological outcomes following haemorrhagic stroke is determined by an increase in the concentration of NO in the CSF of the subject, for example, by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, preferably from about 40% to about 50%, preferably from about 45% to about 55%, preferably from about 50% to about 60%, preferably from about 55% to about 65%, preferably from about 60% to about 70%, preferably from about 65% to about 75%, preferably from about 70% to about 80%, preferably from about 75% to about 85%, preferably from about 80% to about 90%, preferably from about 85% to about 95%, or most preferably from about 90% to 100% when compared to the concentration of NO in the CSF of the subject prior to treatment.

Methods by which the concentration of NO in CSF can be measured will be familiar to persons skilled in the art, an illustrative example of which is described in Pluta et al. (JAMA. 2005; 293(12):1477-1484), the contents of which are incorporated herein by reference in their entirety.

As noted elsewhere herein, the present inventors have surprisingly found that therapeutic Hp can prevent penetration of cell-free Hb from the CSF compartment into the interstitial space of the brain. These surprising findings suggest that compartmentalization of cell-free Hb in the CSF compartments through complexation with Hp can prevent the toxic effects of cell-free Hb (predominantly oxyHb) on the cerebral vasculature and the brain parenchyma. Thus, in an embodiment, the adverse secondary neurological outcome is an adverse secondary neurological outcome within the brain parenchyma.

### Haptoglobin

Haptoglobin (Hp) has a tetrameric structure comprising two alpha and two beta chains, linked by disulphide linkages. The beta chain (245 amino acids) has a mass of about 40 kDa (of which approximately 30% w/w is carbohydrate) and is shared by all phenotypes. The alpha chain exists in at least two forms: alpha1, (83 amino acids, 9 kDa) and alpha2 (142 amino acids, 17.3 kDa). Therefore, Hp occurs as three phenotypes, referred to as Hp1-1, Hp2-1 and Hp2-2. Hp1-1 contains two alpha1 chains, Hp2-2 contains two alpha2 chains, and Hp2-1 contains one alpha1 and one alpha2 chain. Hp 1-1 has a molecular mass of 100 kDa, or 165 kDa when complexed with Hb. Hp1-1 exists as a single isoform, and is also referred to as Hp dimer. Hp2-1 has an average molecular mass of 220 kDa and forms liner polymers. Hp2-2 has an average molecular mass of 400 kDa and forms cyclic polymers. Each different polymeric form is a different isoform. A PCR methodology has been devised (Koch et al. 2002, Clin. Chem. 48: 1377-1382) for studying Hp polymorphism.

Two major alleles, Hp1 and Hp2, exist for the Hp gene found on chromosome 16. The two alleles are responsible for three different possible genotypes with structural polymorphism: homozygous (1-1 or 2-2) and heterozygous 2-1. In Western populations, it is estimated that the distribution of Hp 1-1 is ˜16%, Hp 2-1 is ˜48%, and Hp 2-2 is ˜36%. Hp is cleaved into two subunits a and p chains, joined by a disulphide bond. Both alleles share the same p chain. The p chain is responsible for binding the Hb, thus both genotypes have similar Hb binding affinity.

It is to be understood that naturally-occurring and recombinant forms of Hp are suitable for the methods described herein, as long as they are capable of forming a complex with cell-free Hb and thereby neutralise the biological activity of the cell-free Hb. Suitable naturally-occurring forms of Hp will be known to persons skilled in the art, illustrative examples of which are described in Koch et al. (2002, Clin. Chem. 48: 1377-1382) and Kasvosve et al. (2010, Chapter 2—Haptoglobin Polymorphism and Infection; Advances in Clinical Chemistry, 50:23-46), the entire contents of which are incorporated herein by reference.

In an embodiment, the Hp comprises, consists or consists essentially of plasma derived Hp. The Hp is preferably a human Hp. A variety of protocols for the isolation of Hp from a natural source of Hp- (e.g., blood plasma) will be familiar to persons skilled in the art, illustrative examples of which are described in U.S. Pat. Nos. 4,061,735 and 4,137,307 (to Funakoshi et al.) and US patent publication no. 20140094411 (to Brinkman), the entire contents of which are incorporated herein by reference. Other suitable methods for isolating Hp from a natural source of Hp are described in Katnik & Jadach (1993, Arch. Immunol. Ther. Exp. (Warz) 41: 303-308), Tseng et al. (2004, Protein Expr Purif 33: 265-273), Katnik et al. (1995, Eur J. Clin. Chem. Clin. Biochem. 33:727-732), Yang and Mao (1999, J. Chromatogr. B. Biomed. Sci. Appl., 731: 395-402), and Basler & Burrel (1983 Inflammation 7(4): 387-400).

It is to be understood that the term “haptoglobin”, as used herein, includes all phenotypes (including all isoforms) of Hp. The Hp may be homogenous (insofar as it consists essentially of an Hp of the same isoform) or heterogeneous (insofar as it comprises a combination of different Hp isoforms, including Hp1-1, Hp1-2 and Hp2-2). It is to be understood that the composition of the Hp will ultimately depend on the phenotypes of the source. For example, if pooled plasma samples are used to extract/purify the Hp, more than one isoform of Hp will likely be isolated. Suitable methods for determining Hp isoforms that are present in an isolate will be familiar to persons skilled in the art, illustrative examples of which are discussed in Shih et al. (2014, Hematology, 89(4):443-447), the entire contents of which are incorporated herein by reference. Other suitable methods of determining Hp isoforms that are present in an isolate include high performance size exclusion chromatography (HPLC-SEC assay), Hp ELISA, and turbimetric readings, that different isoforms of Hp will typically give differing signals in the different assays.

In an embodiment, the Hp is selected from the group consisting of an Hp1-1 homodimer, an Hp1-2 multimer, an Hp2-2 multimer and a combination of any of the foregoing. In a preferred embodiment, the Hp comprises, consists or consists essentially of an Hp2-2 multimer. The Hp may be a naturally-occurring Hp (e.g., plasma derived) or it may be produced as a recombinant protein, illustrative examples of which are described elsewhere herein. In an embodiment, the plasma derived Hp comprises, consists or consists essentially of Hp2-2. In another embodiment, the plasma derived Hp comprises, consists or consists essentially of Hp1-1. In a further embodiment, the Hp comprises, consists or consists essentially of recombinant Hp.

Unless stated otherwise, the term Hp, as used herein, includes functional analogues of native or naturally-occurring Hp. The term “functional analogue” is to be understood to mean an agent that shares substantially the same biological activity of naturally-occurring (native) Hp, insofar as that biological activity is at least the ability of the analogue to form a complex with cell-free Hb and thereby neutralise its biological activity. By “substantially the same biological activity” typically means the functional analogue has a binding affinity for cell-free Hb that is at least 40% (e.g., 40%, 45%, 50%, 55%, 60%, 65%, 70%, 85%, 90%, 95%, 100%, 105%, 110%, 115%, 120%, 125%, 130%, 135%, 140%, 145%, 150%, 155%, 160%, 165% and so on) of the binding affinity of naturally-occurring Hp, including naturally-occurring Hp isoforms (e.g., Hp1-1, Hp1-2 and Hp2-2). Suitable methods for determining whether an agent is a functional analogue of Hp will be familiar to persons skilled in the art, illustrative examples of which include are described elsewhere herein (e.g, the ability of the functional analogue to reduce cell-free Hb-induced cerebral vasospasms).

In an embodiment disclosed herein, the functional analogue of Hp is a functional fragment of native Hp. A functional fragment of native Hp can be any suitable length, as long as the fragment retains the ability to form a complex with cell-free Hb and thereby neutralise its biological activity.

In another embodiment, the functional analogue is a peptide that has a different amino acid sequence to a naturally-occurring (native) Hp molecule (i.e., a comparator). The functional analogue may include a molecule that has an amino acid sequence that differs from the amino acid sequence of the alpha and/or beta chains of native Hp by one or more (e.g., 1, 2, 3, 4, 5, 6, 7, 8, 9 or more) amino acid substitutions, wherein said difference does not, or does not completely, abolish the ability of the analogue to form a complex with cell-free Hb and thereby neutralise its biological activity. In some embodiments, the functional analogue comprises amino acid substitutions that enhance the ability of the analogue to form a complex with cell-free Hb, as compared to native Hp. In an embodiment, the functional analogue has an amino acid sequence that differs from the amino acid sequence of the alpha and/or beta chain of native Hp by one or more conservative amino acid substitutions. As used herein, the term “conservative amino acid substitution” refers to changing amino acid identity at a given position to replace it with an amino acid of approximately equivalent size, charge and/or polarity. Examples of natural conservative substitutions of amino acids include the following 8 substitution groups (designated by the conventional one-letter code): (1) M, I, L, V; (2) F, Y, W; (3) K, R, (4) A, G; (5) S, T; (6) Q, N; (7) E, D; and (8) C, S.

In an embodiment, the functional analogue has at least 85% sequence identity to an amino acid sequence of the alpha and/or beta chain of native Hp. Reference to “at least 85%” includes 85%, 86%, 87%, 88%, 89%, 90%, 91%, 92%, 93%, 94%, 95%, 96%, 97%, 98%, 99% or 100% sequence identity or similarity, for example, after optimal alignment or best fit analysis. Thus, in an embodiment, the sequence has at least 85%, preferably at least 86%, preferably at least 87%, preferably at least 88%, preferably at least 89%, preferably at least 90%, preferably at least 91%, preferably at least 92%, preferably at least 93%, preferably at least 94%, preferably at least 95%, preferably at least 96%, preferably at least 97%, preferably at least 98%, preferably at least 99% or preferably 100% sequence identity or sequence homology with the sequences identified herein, for example, after optimal alignment or best fit analysis.

The terms “identity”, “similarity”, “sequence identity”, “sequence similarity”, “homology”, “sequence homology” and the like, as used herein, mean that at any particular amino acid residue position in an aligned sequence, the amino acid residue is identical between the aligned sequences. The term “similarity” or “sequence similarity” as used herein, indicates that, at any particular position in the aligned sequences, the amino acid residue is of a similar type between the sequences. For example, leucine may be substituted for an isoleucine or valine residue. As noted elsewhere herein, this may be referred to as conservative substitution. In an embodiment, an amino acid sequence may be modified by way of conservative substitution of any of the amino acid residues contained therein, such that the modification has no effect on the binding specificity or functional activity of the modified polypeptide when compared to the unmodified (native) Hp polypeptide.

In some embodiments, sequence identity with respect to a peptide sequence relates to the percentage of amino acid residues in the candidate sequence which are identical with the residues of the corresponding peptide sequence after aligning the sequences and introducing gaps, if necessary, to achieve the maximum percentage homology, and not considering any conservative substitutions as part of the sequence identity. Neither N- or C-terminal extensions, nor insertions shall be construed as reducing sequence identity or homology. Methods and computer programs for performing an alignment of two or more amino acid sequences and determining their sequence identity or homology are well known to persons skilled in the art. For example, the percentage of identity or similarity of two amino acid sequences can be readily calculated using algorithms, for example, BLAST, FASTA, or the Smith-Waterman algorithm.

Techniques for determining an amino acid sequence “similarity” are well known to persons skilled in the art. In general, “similarity” means an exact amino acid to amino acid comparison of two or more peptide sequences or at the appropriate place, where amino acids are identical or possess similar chemical and/or physical properties such as charge or hydrophobicity. A so-termed “percent similarity” then can be determined between the compared peptide sequences. In general, “identity” refers to an exact amino acid to amino acid correspondence of two peptide sequences.

Two or more peptide sequences can also be compared by determining their “percent identity”. The percent identity of two sequences may be described as the number of exact matches between two aligned sequences divided by the length of the shorter sequence and multiplied by 100. An approximate alignment for nucleic acid sequences is provided by the local homology algorithm of Smith and Waterman, Advances in Applied Mathematics 2:482-489 (1981). This algorithm can be extended to use with peptide sequences using the scoring matrix developed by Dayhoff (Atlas of Protein Sequences and Structure, M. O. Dayhoff ed., 5 suppl. 3:353-358, National Biomedical Research Foundation, Washington, D.C., USA), and normalized by Gribskov (Nucl. Acids Res. 14(6):6745-6763, 1986). Suitable programs for calculating the percent identity or similarity between sequences are generally known in the art.

Optimal alignment of sequences for aligning a comparison window may be conducted by computerized implementations of algorithms (GAP, BESTFIT, FASTA, and TFASTA in the Wisconsin Genetics Software Package Release 7.0, Genetics Computer Group, 575 Science Drive Madison, Wis., USA) or by inspection and the best alignment (i.e., resulting in the highest percentage homology over the comparison window) generated by any of the various methods selected. Reference also may be made to the BLAST family of programs as for example disclosed by Altschul et al., (1997, Nucl. Acids Res. 25:3389. A detailed discussion of sequence analysis can be found in Unit 19.3 of Ausubel et al. (“Current Protocols in Molecular Biology”, John Wiley & Sons Inc, 1994-1998, Chapter 15).

In an embodiment, a functional analogue includes amino acid substitutions and/or other modifications relative to native Hp in order to increase the stability of the analogue or to increase the solubility of the analogue.

The functional analogue may be a naturally-occurring compound/peptide or it may be synthetically produced by chemical synthesis using methods known to persons skilled in the art.

The Hp may suitably be produced as a recombinant protein in a microorganism, which can be isolated and, if desired, further purified. Suitable microorganisms for the production of recombinant Hp will be familiar to persons skilled in the art, illustrative examples of which include bacteria, yeast or fungi, eukaryote cells (e.g., mammalian or an insect cells), or in a recombinant virus vector (e.g., adenovirus, poxvirus, herpesvirus, Simliki forest virus, baculovirus, bacteriophage, sindbis virus or sendai virus). Suitable bacteria for producing recombinant peptides will be familiar to persons skilled in the art, illustrative examples of which include E. coli, B. subtilis or any other bacterium that is capable of expressing the peptide sequences. Illustrative examples of suitable yeast types for producing recombinant peptides include Saccharomyces cerevisiae, Schizosaccharomyces pombe, Candida, Pichia pastoris or any other yeast capable of expressing peptides. Corresponding methods are well known in the art. Also methods for isolating and purifying recombinantly produced peptide sequences are well known in the art and include, for example, gel filtration, affinity chromatography and ion exchange chromatography.

To facilitate isolation of a recombinant Hp, as described herein, a fusion polypeptide may be made where the peptide sequence of the Hp, or functional analogue thereof, is translationally fused (covalently linked) to a heterologous polypeptide which enables isolation by affinity chromatography. Illustrative examples of suitable heterologous polypeptides are His-Tag (e.g. Hiss. 6 histidine residues), GST-Tag (Glutathione-S-transferase) etc.

For preparing recombinant Hp, phage libraries and/or peptide libraries are also suitable, for instance, produced by means of combinatorial chemistry or obtained by means of high throughput screening techniques for the most varying structures (see, for example, Display: A Laboratory Manual by Carlos F. Barbas (Editor), et al.; and Willats W G Phage display: practicalities and prospects. Plant Mol. Biol. 2002 December; 50(6):837-54).

Illustrative examples of recombinant Hp include the peptides with accession no. NP_005134 (as described by Morishita et al. 2018, Clin. Chim. Acta 487, 84-89) and accession no. P00738.

The Hp may be fused, coupled or otherwise attached to one or more heterologous moieties as part of a fusion protein. The one or more heterologous moieties may improve, enhance or otherwise extend the activity or stability of the Hp. In an embodiment, the Hp, as described herein, is suitably attached to a heterologous moiety for extending the half-life of the Hp in vivo. Suitable half-life extending heterologous moieties will be familiar to persons skilled in the art, illustrative examples of which include polyethylene glycol (PEGylation), glycosylated PEG, hydroxyl ethyl starch (HESylation), polysialic acids, elastin-like polypeptides, heparosan polymers and hyaluronic acid. Thus, in an embodiment disclosed herein, heterologous moiety is selected from the group consisting of polyethylene glycol (PEGylation), glycosylated PEG, hydroxyl ethyl starch (HESylation), polysialic acids, elastin-like polypeptides, heparosan polymers and hyaluronic acid. In other embodiments, the heterologous moiety may be a heterologous amino acid sequence fused to the Hp.

Alternatively, or in addition, the heterologous moiety may be chemically conjugated to the Hp, for example, a covalent bond. The half-life extending heterologous moiety can be fused, conjugated or otherwise attached to the Hp by any suitable means known to persons skilled in the art, an illustrative example of which is via a chemical linker. The principle of this conjugation technology has been described in an exemplary manner by Conjuchem LLC (see, e.g., U.S. Pat. No. 7,256,253), the entire contents of which are incorporated herein by reference.

In other embodiments, the heterologous moiety is a half-life enhancing protein (HLEP). Suitable half-life enhancing proteins will be familiar to persons skilled in the art, an illustrative example of which includes albumin or fragments thereof. Thus, in an embodiment, the HLEP is an albumin or a fragment thereof. The N-terminus of the albumin or fragment thereof may be fused to the C-terminus of the alpha and/or beta chains of the Hp. Alternatively, or in addition, the C-terminus of the albumin or fragment thereof may be fused to the N-terminus of the alpha and/or beta chains of the Hp. One or more HLEPs may be fused to the N- or C-terminal part(s) of the alpha and/or beta chains of the Hp provided that they do not abolish the binding of the Hp to cell-free Hb. It is to be understood, however, that some reduction in the binding of the Hp to cell-free Hb may be acceptable, as long as the Hp component of the fusion protein is still capable of forming a complex with, and thereby neutralise, cell-free Hb.

The fusion protein may further comprise a chemical bond or a linker sequence positioned between the Hp and the heterologous moiety. The linker sequence may be a peptidic linker consisting of one or more amino acids, in particular of 1 to 50, preferably 1 to 30, preferably 1 to 20, preferably 1 to 15, preferably 1 to 10, preferably 1 to 5 or more preferably 1 to 3 (e.g. 1, 2 or 3) amino acids and which may be equal or different from each other. Preferably, the linker sequence is not present at the corresponding position in the wild-type Hp.

Preferred amino acids present in said linker sequence include Gly and Ser. In a preferred embodiment, the linker sequence is substantially non-immunogenic to the subject to be treated in accordance with the methods disclosed herein. By substantially non-immunogenic is meant that the linker sequence will not raise a detectable antibody response to the linker sequence in the subject to which it is administered. Preferred linkers may be comprised of alternating glycine and serine residues. Suitable linkers will be familiar to persons skilled in the art, illustrative examples of which are described in WO2007/090584. In an embodiment, the peptidic linker between the Hp and the heterologous moiety comprises, consists or consists essentially of peptide sequences, which serve as natural interdomain linkers in human proteins. Such peptide sequences in their natural environment may be located close to the protein surface and are accessible to the immune system so that one can assume a natural tolerance against this sequence. Illustrative examples are given in WO 2007/090584. Suitable cleavable linker sequences are described, e.g., in WO 2013/120939 A1.

Illustrative examples of suitable HLEP sequences are described infra. Likewise disclosed herein are fusions to the exact “N-terminal amino acid” or to the exact “C-terminal amino acid” of the respective HLEP, or fusions to the “N-terminal part” or “C-terminal part” of the respective HLEP, which includes N-terminal deletions of one or more amino acids of the HLEP. The fusion protein may comprise more than one HLEP sequence, e.g. two or three HLEP sequences. These multiple HLEP sequences may be fused to the C-terminal part of the alpha and/or beta chains of the Hp in tandem, e.g. as successive repeats.

In an embodiment, the heterologous moiety is a half-life extending polypeptide. In an embodiment, the half-life extending polypeptide is selected from the group consisting of albumin, a member of the albumin-family or fragments thereof, solvated random chains with large hydrodynamic volume (e.g. XTEN (see Schellenberger et al. 2009; Nature Biotechnol. 27:1186-1190), homo-amino acid repeats (HAP) or proline-alanine-serine repeats (PAS), afamin, alpha-fetoprotein, Vitamin D binding protein, transferrin or variants or fragments thereof, carboxyl-terminal peptide (CTP) of human chorionic gonadotropin-β subunit, a polypeptide capable of binding to the neonatal Fc receptor (FcRn), in particular an immunoglobulin constant region and portions thereof, e.g. the Fc fragment, polypeptides or lipids capable of binding under physiological conditions to albumin, to a member of the albumin-family or to fragments thereof or to an immunoglobulin constant region or portions thereof. The immunoglobulin constant region or portions thereof is preferably an Fc fragment of immunoglobulin G1 (IgG1), an Fc fragment of immunoglobulin G2 (IgG2) or an Fc fragment of immunoglobulin A (IgA). A half-life enhancing polypeptide, as used herein, may be a full-length half-life-enhancing protein or one or more fragments thereof that are capable of stabilizing or prolonging the therapeutic activity or the biological activity of the Hp, in particular of increasing the in vivo half-life of the Hp. Such fragments may be of 10 or more amino acids in length or may include at least about 15, preferably at least about 20, preferably at least about 25, preferably at least about 30, preferably at least about 50, or more preferably at least about 100, or more contiguous amino acids from the HLEP sequence, or may include part or all of specific domains of the respective HLEP, as long as the HLEP fragment provides a functional half-life extension of at least 10%, preferably of at least 20%, or more preferably of at least 25%, compared to the respective Hp in the absence of the HLEP. Methods of determining whether a heterologous moiety provides a functional half-life extension to the Hp (in vivo or in vitro) will be familiar to persons skilled in the art, illustrative examples of which are described elsewhere herein.

The HLEP portion of the fusion protein, as descried herein, may be a variant of a wild type HLEP. The term “variant” includes insertions, deletions and/or substitutions, either conservative or non-conservative, where such changes do not substantially alter the ability of the Hp to form a complex with, and thereby neutralise, cell-free Hb. The HLEP may suitably be derived from any vertebrate, especially any mammal, for example human, monkey, cow, sheep, or pig. Non-mammalian HLEPs include, but are not limited to, hen and salmon.

The fusion proteins, as described herein, can be created by in-frame joining of at least two DNA sequences encoding the Hp and the heterologous moiety, such as a HLEP. Persons skilled in the art will understand that translation of the fusion protein DNA sequence will result in a single protein sequence. As a result of an in-frame insertion of a DNA sequence encoding a peptidic linker according to an embodiment disclosed herein, a fusion protein comprising the Hp, a suitable linker and the heterologous moiety can be obtained.

In an embodiment disclosed herein, the Hp is fused to a heterologous moiety. In an embodiment, the heterologous moiety comprises, consists or consists essentially of a polypeptide selected from the group consisting of albumin or fragments thereof, transferrin or fragments thereof, the C-terminal peptide of human chorionic gonadotropin, an XTEN sequence, homo-amino acid repeats (HAP), proline-alanine-serine repeats (PAS), afamin, alpha-fetoprotein, Vitamin D binding protein, polypeptides capable of binding under physiological conditions to albumin or to immunoglobulin constant regions, polypeptides capable of binding to the neonatal Fc receptor (FcRn), particularly immunoglobulin constant regions and portions thereof, preferably the Fc portion of immunoglobulin, and combinations of any of the foregoing. In another embodiment, the heterologous moiety is selected from the group consisting of hydroxyethyl starch (HES), polyethylene glycol (PEG), polysialic acids (PSAs), elastin-like polypeptides, heparosan polymers, hyaluronic acid and albumin binding ligands, e.g. fatty acid chains, and combinations of any of the foregoing.

The terms, “human serum albumin” (HSA) and “human albumin” (HA) and “albumin” (ALB) are used interchangeably herein. The terms “albumin” and “serum albumin” are broader, and encompass human serum albumin (and fragments and variants thereof), as well as albumin from other species (and fragments and variants thereof).

As used herein, “albumin” refers collectively to albumin polypeptide or amino acid sequence, or an albumin fragment or variant, having one or more functional activities (e.g., biological activities) of albumin. In particular, “albumin” refers to human albumin or fragments thereof, including the mature form of human albumin or albumin from other vertebrates or fragments thereof, or analogs or variants of these molecules or fragments thereof. In some embodiments disclosed herein, the alternative term “FP” is used to identify the HLEP, in particular to define albumin as the HLEP.

The fusion proteins described herein may suitably comprise naturally-occurring polymorphic variants of human albumin and/or fragments of human albumin. Generally speaking, an albumin fragment or variant will be at least 10, preferably at least 40, or most preferably more than 70 amino acids in length.

In an embodiment, the HLEP is an albumin variant with enhanced binding to the FcRn receptor. Such albumin variants may lead to a longer plasma half-life of the Hp or functional analogue thereof compared to the Hp or functional fragment thereof that is fused to a wild-type albumin. The albumin portion of the fusion proteins described herein may suitably comprise at least one subdomain or domain of human albumin or conservative modifications thereof.

In an embodiment, the heterologous moiety is an immunoglobulin molecule or a functional fragment thereof. Immunoglobulin G (IgG) constant regions (Fc) are known in the art to increase the half-life of therapeutic proteins (see, e.g., Dumont J A et al. 2006. BioDrugs 20:151-160). The IgG constant region of the heavy chain consists of 3 domains (CH1-CH3) and a hinge region. The immunoglobulin sequence may be derived from any mammal, or from subclasses IgG1, IgG2, IgG3 or IgG4, respectively. IgG and IgG fragments without an antigen-binding domain may also be used as a heterologous moiety, including as a HLEP. The Hp or functional analogue thereof may suitably be connected to the IgG or the IgG fragments via the hinge region of the antibody or a peptidic linker, which may even be cleavable. Several patents and patent applications describe the fusion of therapeutic proteins to immunoglobulin constant regions to enhance the therapeutic proteins' in vivo half-lives. For example, US 2004/0087778 and WO 2005/001025 describe fusion proteins of Fc domains or at least portions of immunoglobulin constant regions with biologically active peptides that increase the half-life of the peptide, which otherwise would be quickly eliminated in vivo. Fc-IFN-β fusion proteins were described that achieved enhanced biological activity, prolonged circulating half-life and greater solubility (WO 2006/000448 A2). Fc-EPO proteins with a prolonged serum half-life and increased in vivo potency were disclosed (WO 2005/063808 A1) as well as Fc fusions with G-CSF (WO 2003/076567 A2), glucagon-like peptide-1 (WO 2005/000892 A2), clotting factors (WO 2004/101740 A2) and interleukin-10 (U.S. Pat. No. 6,403,077), all with half-life enhancing properties.

Illustrative examples of suitable HLEP which can be used in accordance with the present invention are also described in WO 2013/120939 A1, the contents of which are incorporated herein by reference in their entirety.

The term “therapeutically effective amount”, as used herein, means the amount or concentration of Hp in the CSF that is sufficient to allow the Hp to bind to, and form a complex with, cell-free Hb present in the CSF and thereby neutralise the otherwise adverse biological effect of the cell-free Hb. It would be understood by persons skilled in the art that the therapeutically effective amount of peptide may vary depending upon several factors, illustrative examples of which include whether the Hp is to be administered directly to the subject (e.g, intrathecally, intracranially or intracerebroventricularly), the health and physical condition of the subject to be treated, the taxonomic group of subject to be treated, the severity of the haemorrhage (e.g., the extent of bleeding), the route of administration, the concentration and/or amount of cell-free Hb in the CSF compartment and combinations of any of the foregoing.

The therapeutically effective amount of Hp will typically fall within a relatively broad range that can be determined by persons skilled in the art. Illustrative examples of a suitable therapeutically effective amounts of Hp include from about 2 μM to about 20 mM, preferably from about 2 μM to about 5 mM, preferably from about 100 μM to about 5 mM, preferably from about 2 μM to about 300 μM, preferably from about 5 μM to about 100 μM, preferably from about 5 μM to about 50 μM, or more preferably from about 10 μM to about 30 μM.

In an embodiment, the therapeutically effective amount of Hp is from about 2 μM to about 20 mM. In an embodiment, the therapeutically effective amount of Hp is from about 2 μM to about 5 mM. In an embodiment, the therapeutically effective amount of Hp is from about 100 μM to about 5 mM. In an embodiment, the therapeutically effective amount of Hp is from about 2 μM to about 300 μM. In an embodiment, the therapeutically effective amount of Hp is from about 5 μM to about 50 μM. In an embodiment, the therapeutically effective amount of Hp is from about 10 μM to about 30 μM.

In an embodiment, the therapeutically effective amount of Hp is at least an equimolar amount to the concentration of cell-free Hb in the CFS of the subject following the haemorrhage. In another embodiment, the therapeutically effective amount of Hp is an amount sufficient to complex from about 3 μM to about 300 μM cell-free Hb in CSF. Suitable methods of measuring the concentration of cell-free Hb in CSF will be known to persons skilled in the art, illustrative examples of which are described in Cruickshank A M., 2001, ACP Best Practice No 166, J. Clin. Path., 54(11):827-830) and Hugelshofer M. et al., 2018. World Neurosurg.; 120:e660-e666), the contents of which are incorporated herein by reference in their entirety.

It is to be understood that achieving a therapeutically effective amount of Hp, as herein described, may depend on the final volume of the CSF to which the Hp, or functional analogue thereof, is exposed. For example, where the Hp is to administered to an adult human subject (e.g., intrathecally), and taking into account that the average volume of CSF in an adult human subject is about 150 mL, a therapeutically effective amount of about 10 μM Hp can be achieved by administering to the subject a 5 mL solution of about 310 μM Hp. In another illustrative example, the methods described herein comprise removing 50 mL of CSF from the subject and replacing it with 50 mL of artificial CSF comprising about 30 μM Hp, thereby achieving a therapeutically effective amount of about 10 μM Hp in the CSF compartment of the subject.

Dosages of Hp may also be adjusted to provide the optimum therapeutic response. For example, several divided doses may be administered daily, weekly, or other suitable time intervals, or the dosages may be proportionally reduced as indicated by the exigencies of the situation.

The term “exposing”, as used herein, means bringing into contact the CSF with the Hp in such a way as to allow the Hp to bind to and form a complex with cell-free Hb (the predominant form of which will be oxyHb) that is present in the CSF, whereby the formation of Hb:Hp complexes substantially neutralises the otherwise adverse biological effect of cell-free Hb on brain tissue. By “substantially neutralise” is meant a reduction to the adverse biological effect of cell-free Hb on brain tissue, as represented subjectively or qualitatively as a percentage reduction by at least 10%, preferably from about 10% to about 20%, preferably from about 15% to about 25%, preferably from about 20% to about 30%, preferably from about 25% to about 35%, preferably from about 30% to about 40%, preferably from about 35% to about 45%, preferably from about 40% to about 50%, preferably from about 45% to about 55%, preferably from about 50% to about 60%, preferably from about 55% to about 65%, preferably from about 60% to about 70%, preferably from about 65% to about 75%, preferably from about 70% to about 80%, preferably from about 75% to about 85%, preferably from about 80% to about 90%, preferably from about 85% to about 95%, or most preferably from about 90% to 100% comparted to the biological effect of cell-free Hb on brain tissue in the absence of therapeutic Hp, including by at least 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%. Methods by which a reduction to the adverse biological effect of cell-free Hb can be measured or determined (qualitatively or quantitatively) will be familiar to persons skilled in the art, illustrative examples of which are described elsewhere herein.

As also noted herein, the present inventors have also shown, for the first time, that a therapeutically effective amount of Hp can prevent interference of cell-free oxyHb in CSF with cerebrovascular NO-signalling to reduce the incidence of vasospasms and DIND. Using dynamic MRI, the present inventors confirmed a rapid, macroscopically identical dispersion of Hb and Hb:Hp complexes in the subarachnoid space after injection into the ventricular system. Histological analysis also unexpectedly revealed extensive penetration of Hb from the CSF compartment into the interstitial space of the brain, whereas Hb:Hp complexes were largely confined to the subarachnoid space. These surprising findings suggest that compartmentalization of cell-free Hb in the CSF compartments through complexation with Hp can prevent the toxic effects of cell-free Hb (predominantly oxyHb) on the cerebral vasculature and the brain parenchyma.

It is to be understood that the manner in which the CSF of a subject in need thereof is exposed to a therapeutically effective amount of Hp, as described herein, may vary, depending, for example, on whether said exposure is to be performed within the CSF compartment of a subject in need thereof (i.e., in vivo) or extracorporeally in CSF obtained from the subject (i.e, ex vivo). Where said exposure is to be performed within the CSF compartment of a subject in need thereof (i.e., in vivo), the route of administration of the Hp will be selected to allow the Hp to contact cell-free Hb within the CSF compartment. Suitable routes of administration will be familiar to persons skilled in the art, illustrative examples of which include intrathecal, intracranial and intracerebroventricular. In an embodiment, the therapeutically effective amount of Hp is administered via an external ventricular drain that is placed, for example, in the subject after a haemorrhagic stroke to temporarily drain the CSF and decrease intracranial pressure.

In an embodiment, the method comprises intracranially administering to the subject the therapeutically effective amount of the Hp.

In an embodiment, the method comprises intrathecally administering to the subject the therapeutically effective amount of the Hp. In an embodiment, the method comprises intrathecally administering to the subject the therapeutically effective amount of the Hp into the spinal canal. In an embodiment, the method comprises intrathecally administering to the subject the therapeutically effective amount of the Hp into the subarachnoid space. In an embodiment, the method comprises intracerebroventricularly administering to the subject the therapeutically effective amount of the Hp.

Alternatively, or in addition, the methods described herein comprise removing CSF from a subject in need thereof, exposing the CSF to a therapeutically effective amount of Hp for a period of time sufficient to allow the Hp to form a complex with, and thereby neutralise, the cell-free Hb in the CSF, removing the Hb:Hp complexes thus formed in the CSF to produce an Hb-diminished CSF and administering the Hb-diminished CSF to the subject (e.g, intrathecally, or intracerebroventricularly). It will be understood that removing CSF from the CSF compartment will typically not result in a CSF compartment that is entirely free of CSF, noting that at least some CSF will remain in the CSF compartment. Optionally, the CSF compartment can be rinsed with a pharmaceutically acceptable wash solution once CSF has been removed in order to remove at least some of the residual Hb that may be present in the CSF compartment. The wash solution may optionally comprise Hp, to further complex and thereby neutralise at least some of the residual cell-free Hb that may be present in the CSF compartment. In an embodiment, the wash solution is an artificial CSF, as described elsewhere herein.

In an embodiment, the method described herein comprises removing a sample of CSF from the CSF compartment of a subject in need thereof, adding Hp to the CSF sample to obtain an Hp-enriched CSF sample, administering the Hp-enriched CSF sample to the CSF compartment of the subject, thereby exposing the CSF compartment to a therapeutically effective amount of the Hp and for a period of time sufficient to allow the Hp to form a complex with, and thereby neutralise, cell-free Hb in the CSF of the subject, and optionally, repeating the above steps. Preferably, the amount of Hp that is added to the CSF sample will be determined such that, upon administration to the subject, will provide a therapeutically effective amount of Hp within the CSF of the subject. Hence, the amount of Hp to be added in the CSF sample will depend on the volume of CSF sample that is removed and re-administered to the subject.

In an embodiment, the method comprises:
(i) obtaining sample of CSF from the CSF compartment of the subject following the haemorrhage;
(ii) adding to the CSF sample of step (i) Hp to obtain an Hp-enriched CSF sample;
(iii) administering the Hp-enriched CSF sample to the subject, thereby exposing the CSF compartment of the subject to a therapeutically effective amount of Hp for a period of time sufficient to allow the Hp to form a complex with cell-free Hb in the CSF compartment of the subject; and
(iv) optionally repeating steps (i) to (iii).

The volume of CSF that is to be removed and re-administered to the subject will desirably be substantially the same. For example, if a 50 mL sample of CSF is removed from the CSF compartment of the subject, the entire 50 mL volume of CSF comprising the Hp will be re-administered to the subject. However, it will be understood that the volumes may be dissimilar, as long as any difference in volumes does not give rise to significant adverse clinical outcomes. In some embodiments, the volume of CSF that is re-administered to the subject will be less than the volume of CSF that was removed from the subject. In other embodiments, the volume of CSF that is re-administered to the subject will be greater than the volume of CSF that was removed from the subject, with the addition of Hp, alone or in combination with any other therapeutical agents, making up the extra volume.

In another embodiment, the method described herein comprises removing a volume of CSF from the CSF compartment of a subject in need thereof, replacing the volume of CSF removed from the subject with a volume of artificial CSF comprising a Hp, thereby exposing the CSF of the subject to a therapeutically effective amount of the Hp and for a period of time sufficient to allow the Hp to form a complex with, and thereby neutralise, cell-free Hb in the CSF of the subject. Preferably, the amount of Hp in the artificial CSF will be determined such that, upon administration to the subject, will provide a therapeutically effective amount of Hp within the CSF of the subject. Hence, the amount of Hp to be added to the artificial CSF will depend on the volume of artificial CSF that will be administered to the subject.

In an embodiment, the method comprises:
(i) removing a volume of CSF from the CSF compartment of the subject following the haemorrhage;
(ii) providing an artificial CSF comprising Hp;
(iii) administering the artificial CSF of (ii) to the subject, thereby exposing the CSF compartment of the subject to a therapeutically effective amount of Hp for a period of time sufficient to allow the Hp to form a complex with cell-free Hb in the CSF compartment of the subject; and
(iv) optionally repeating steps (i) to (iii).

The volume of artificial CSF that is administered to the subject will desirably be substantially the same as the volume of CSF removed from the subject. For example, if a 50 mL sample of CSF is removed from the CSF compartment of the subject, a volume of about 50 mL of artificial CSF comprising the therapeutically effective amount of Hp will be used to replace the volume of CSF removed. However, it will be understood that the volumes may be dissimilar, as long as any difference in volumes does not give rise to significant adverse clinical outcomes. In some embodiments, the volume of artificial CSF that is administered to the subject will be less than the volume of CSF that was removed from the subject. In other embodiments, the volume of artificial CSF that is administered to the subject will be greater than the volume of CSF that was removed from the subject.

In an embodiment, the method described herein comprises exposing the CSF to the Hp within about 21 days after the haemorrhagic stroke. In another embodiment disclosed herein, the method comprises exposing the CSF to the Hp from about 2 days to about 4 days after the haemorrhagic stroke. In yet another embodiment, the method comprises exposing the CSF to the Hp from about 5 days to about 14 days after the haemorrhagic stroke.

The present inventors have surprisingly shown that exposing cell-free Hb within the subarachnoid space to a therapeutically effective amount of Hp for a period of at least about 2 minutes is sufficient to form detectable Hb:Hp complexes in the CSF. Thus, in an embodiment, the period of time to which the CSF is exposed to the therapeutically effective amount of Hp is at least about 2 minutes (e.g., 2, 3, 4, 5, 6, 7, 8, 9, 10, 11, 12, 13, 14 minutes, and so on). In an embodiment, the period of time to which the CSF is exposed to the therapeutically effective amount of Hp is at least about 4 minutes. In another embodiment, the period of time to which the CSF is exposed to the therapeutically effective amount of Hp is at least about 5 minutes. In yet another embodiment, the period of time to which the CSF is exposed to the therapeutically effective amount of Hp is at least about 10 minutes. In an embodiment, the period of time to which the CSF is exposed to the therapeutically effective amount of Hp is from about 2 minutes to about 45 minutes, preferably from about 2 minutes to about 20 minutes, or more preferably from about 4 minutes to about 10 minutes.

The term “subject”, as used herein, refers to a mammalian subject for whom treatment or prophylaxis is desired. Illustrative examples of suitable subjects include primates, especially humans, companion animals such as cats and dogs and the like, working animals such as horses, donkeys and the like, livestock animals such as sheep, cows, goats, pigs and the like, laboratory test animals such as rabbits, mice, rats, guinea pigs, hamsters and the like and captive wild animals such as those in zoos and wildlife parks, deer, dingoes and the like. In an embodiment, the subject is a human. In a further embodiment, the subject is a paediatric patient aged (i) from birth to about 2 years of age, (ii) from about 2 to about 12 years of age or (iii) from about 12 to about 21 years of age.

In an embodiment, the methods described herein comprise exposing the CSF to the therapeutically effective amount of Hp extracorporeally.

Where the CSF is to be exposed to the Hp extracorporeally (ex vivo), the therapeutically effective amount may depend on the volume of CSF to which the Hp will be expose, whether the CSF is exposed to an Hp that is in solution or immobilise on a substrate (e.g., for affinity chromatography) and combinations of any of the foregoing. For instance, where the CSF is to be exposed to the Hp for complexation and removal of cell-free Hb by affinity chromatography, the amount of Hp that is immobilise on the substrate need not result in complete complexation of cell-free Hb during an initial pass, and it may be that multiple passes over the substrate may be required to complex and thereby remove substantially all of the cell-free Hb from the CSF.

In an embodiment, the method comprises (i) obtaining a CSF sample from the subject following a haemorrhagic stroke and prior to exposing the CSF to the Hp; (ii) measuring the amount of cell-free Hb in the CSF sample obtained in step (i); and (iii) determining the at least equimolar amount of Hp based on the concentration of cell-free Hb from step (ii). Suitable methods of measuring the amount of cell-free Hb in a CSF sample will be known to persons skilled in the art, an illustrative example of which is described by Oh et al. (2016, Redox Biology, 9: 167-177), the contents of which are incorporated herein by reference in their entirety.

As noted elsewhere herein, complexation of cell-free Hb with the Hb within CSF will neutralise the otherwise adverse biological activity of cell-free Hb on brain tissue. Whilst it is generally unnecessary to extract or remove cell-free Hb:Hp complexes that have formed in accordance with the methods disclosed herein, it may be desirable in some instances to remove said complexes. For example, where the methods described herein comprise removing CSF from the subject and subsequently exposing the CSF to Hp extracorporeally, it may be desirable to remove cell-free Hb:Hp complexes that have formed in the CSF prior to re-administering the CSF to the subject. Thus, in some embodiments, the method comprises removing Hp:cell-free Hb complexes formed in the CSF.

In an embodiment, the method comprises:
(i) obtaining CSF from the subject following the haemorrhage;
(ii) exposing the CSF from step (i) to the Hp under conditions to allow the Hp to form a complex with cell-free Hb in the CSF;
(iii) extracting the Hp:cell-free Hb complexes from the CSF following step (ii) to obtain an Hb-diminished CSF that has a lower amount of cell-free Hb when compared to the CSF from step (i);
(iv) optionally repeating steps (ii) and (iii) to obtain an Hb-diminished CSF that is substantially free of cell-free Hb; and
(v) administering the Hb-diminished CSF obtained from step (iii) or step (iv) to the CSF compartment of the subject.

As used herein, an “Hb-diminished CSF” means CSF from which an amount of cell-free Hb has been removed such that the CSF has a lower amount of cell-free Hb when compared to the amount of cell-free Hb prior to step (iii). It is to be understood that the term “Hb-diminished CSF” is not intended to imply that all of the cell-free Hb has been removed from the CSF and therefore includes embodiments in which at least some cell-free Hb. In an embodiment, the Hb-diminished CSF comprises at least about 5%, preferably at least about 10%, preferably at least about 15%, preferably at least about 20%, preferably at least about 25%, preferably at least about 30%, preferably at least about 35%, preferably at least about 40%, preferably at least about 45%, preferably at least about 50%, preferably at least about 55%, preferably at least about 60%, preferably at least about 65%, preferably at least about 70%, preferably at least about 75%, preferably at least about 80%, preferably at least about 85%, preferably at least about 90%, or more preferably at least about 95% less cell-free Hb when compared to the amount of cell-free Hb in the CSF obtained from the subject. In an embodiment, the Hb-diminished CSF comprises from about 5% to about 10%, preferably from about 10% to about 20%, preferably from about 20% to about 30%, preferably from about 30% to about 40%, preferably from about 40% to about 50%, preferably from about 50% to about 60%, preferably from about 60% to about 70%, preferably from about 70% to about 80%, preferably from about 80% to about 90%, or more preferably from about 90% to about 99% less cell-free Hb when compared to the amount of cell-free Hb in the CSF obtained from the subject.

As described elsewhere herein, the Hb-diminished CSF may optionally be further treated by repeating steps (ii) and (iii) to remove additional cell-free Hb from the CSF, preferably to obtain an Hb-diminished CSF that is substantially free of cell-free Hb. By “substantially free of cell-free Hb” means the Hb-diminished CSF comprises from about 70% to about 80%, preferably from about 80% to about 90%, or more preferably from about 90% to about 99% less cell-free Hb when compared to the amount of cell-free Hb in the CSF obtained from the subject.

In an embodiment, the method comprises repeating steps (ii) and (iii) at least once (e.g., 1, 2, 3, 4, 5, 6, 7, 8, 9, 10, 11 times, and so on). In an embodiment, the method comprises repeating steps (ii) and (iii) at least 1 time, preferably 2 times, preferably 3 times, preferably 4 times, preferably 5 times, preferably 6 times, preferably 7 times, preferably 8 times, preferably 9 times, or more preferably 10 times, as required to obtain an Hb-diminished CSF that is substantially free of cell-free Hb.

It is to be understood that the number of times that steps (ii) and (iii) need to be repeated to obtain an Hb-diminished CSF that is substantially free of cell-free Hb may depend on several factors, including (but not limited to) the concentration of the cell-free Hb in the CSF from the subject, the concentration of the Hb that is employed, the method of extraction, and so forth. In some instances, it may be desirable to perform steps (ii) and (iii) only once, in particular where repeating steps (ii) and (iii) may expose the CSF to contaminants, such as bacteria, yeast, fungus and viruses.

Suitable methods of extracting Hp:cell-free Hb complexes from CSF will be known to persons skilled in the art, illustrative examples of which include size exclusion chromatography and/or affinity chromatography. Size exclusion chromatography allows Hp:cell-free Hb complexes to be identified and separated from other components in the CSF by virtue of their larger size relative to free Hb and Hp. Affinity chromatography allows Hp:cell-free Hb complexes to be identified and separated from other components in the CSF by using a binding agent that binds specifically to an Hb:Hp complex with negligible binding to free Hb. Suitable binding agents, include antibodies or anti-binding fragments thereof, as would be familiar to persons skilled in the art.

In an embodiment, step (ii) comprises passing the CSF from step (i) over a substrate to which the Hp is immobilised. This advantageously allows the cell-free Hb in the CSF to bind to the Hp thereby mobilising the cell-free Hb to the substrate and allowing the Hb-diminished CSF to be conveniently eluted from the substrate. Thus, in an embodiment, the Hp in step (ii) is immobilised on a substrate. Suitable substrates will be familiar to persons skilled in the art, illustrative examples of which include a size exclusion chromatography resin and affinity chromatography resin. In an embodiment, the substrate is an affinity chromatography resin.

In an embodiment, step (ii) comprises passing the CSF from step (i) through an affinity chromatography resin under conditions that allow the cell-free Hb in the CSF to bind to the resin; wherein step (iii) comprises eluting the CSF from the resin following step (ii); and wherein step (iv) comprising recovering the eluted CSF.

Prior to administering the Hb-diminished CSF obtained from step (iii) or step (iv) to the CSF compartment of the subject in step (vi), it may be desirable to add to the Hb-diminished CSF a therapeutically effective amount of Hp in order to scavenge residual cell-free Hb that may be present in the subject's CSF compartment and could give rise to an adverse secondary neurological outcome, as described elsewhere herein. Thus, in an embodiment, the method comprises adding to the Hb-diminished CSF prior to step (v) a therapeutically effective amount of Hp, as described elsewhere herein.

Persons skilled in the art will understand that once CSF has been removed from the CSF compartment of a subject following a haemorrhagic stroke, there may be residual cell-free Hb remaining in the CSF compartment. It may therefore be desirable to rinse the CSF compartment (once the CSF has been removed in accordance with step (i), above) in order to remove at least some of the residual cell-free Hb and thereby eliminate or otherwise reduce the adverse secondary neurological outcomes to which such residual cell-free Hb may otherwise give rise. Thus, in an embodiment, the method further comprises washing the CSF compartment following step (i) with a wash solution.

Suitable wash solutions will be familiar to persons skilled in the art. In an embodiment, the wash solution is an artificial CSF.

Artificial cerebrospinal fluid (aCSF) is typically a fluid that mimics natural CSF, including by salt content. Suitable compositions of aCSF will be familiar to persons skilled in the art, illustrative examples of which are described in US 2006/0057065 and Matzneller et al. (Pharmacology, 2016; 97(5-6):233-44), the contents of which are incorporated herein by reference in their entirety. The aCSF may comprise NaCl at a similar concentration to that found in natural CSF, as will be familiar to persons skilled in the art, and would typically include concentrations within about 15%, more preferably within about 10% of the concentration of NaCl in natural CSF. The aCSF may comprise NaHCO₃ at a similar concentration to that found in natural CSF, as will be familiar to persons skilled in the art, and would typically include concentrations within about 15%, more preferably within about 10% of the concentration of NaHCO₃ in natural CSF. The aCSF may comprise KCl at a similar concentration to that found in natural CSF, as will be familiar to persons skilled in the art, and would typically include concentrations within about 15%, more preferably within about 10% of the concentration of KCl in natural CSF. The aCSF may comprise NaH₂PO₄ at a similar concentration to that found in natural CSF, as will be familiar to persons skilled in the art, and would typically include concentrations within about 15%, more preferably within about 10% of the concentration of NaH₂PO₄ in natural CSF. The aCSF may comprise MgCl₂ at a similar concentration to that found in natural CSF, as will be familiar to persons skilled in the art, and would typically include concentrations within about 15%, more preferably within about 10% of the concentration of MgCl₂ in natural CSF. The aCSF may comprise glucose at a similar concentration to that found in natural CSF, as will be familiar to persons skilled in the art, and would typically include concentrations within about 15%, more preferably within about 10% of the concentration of glucose in natural CSF. Alternatively, the artificial CSF may omit glucose so as to reduce the likelihood of bacterial growth in any catheter used to administer the aCSF to a subject.

In an embodiment, the artificial CSF/wash solution comprises NaCl, KCl, KH₂PO₄, NaHCO₃, MgCllH₂O, CaCl₂H₂O and glucose.

In an embodiment, the wash solution comprises Hp.

In an embodiment, the wash solution comprises from about 2 μM to about 20 mM Hp. In an embodiment, the wash solution comprises from about 2 μM to about 5 mM Hp. In an embodiment, the wash solution comprises from about 100 μM to about 5 mM Hp. In an embodiment, the wash solution comprises from about 2 μM to about 300 μM Hp. In an embodiment, the wash solution comprises from about 5 μM to about 50 μM Hp. In an embodiment, the wash solution comprises from about 10 μM to about 30 μM Hp.

In an embodiment, the wash solution comprises at least an equimolar amount of Hp to the concentration of cell-free Hb in the CFS of the subject following the haemorrhage. In another embodiment, the wash solution comprises from about 3 μM to about 300 μM Hp.

In some embodiments, it may be desirable to forego steps of extracorporeally removing cell-free Hb from the CSF of a subject, as described herein, and instead replace the subject's CSF with an artificial CSF, as described elsewhere herein. Thus, in an embodiment, the method comprises:
(i) removing CSF from the subject following the haemorrhage;
(ii) rinsing the CSF compartment of the subject following step (i) with a wash solution comprising a therapeutically effective amount of Hp;
(iii) optionally repeating step (ii); and
(iv) administering to the CSF compartment of the subject, following step (ii) or step (iii), an artificial CSF.

By incorporating a therapeutically effective amount of Hp into the wash solution, residual cell-free Hb in the CSF compartment can be complexed with the Hp, thereby neutralising the adverse biological effect of cell-free Hb on brain tissue.

In an embodiment, the artificial CSF comprises NaCl, KCl, KH₂PO₄, NaHCO₃, MgCl₆H₂O, CaCl₂H₂O and glucose. In an embodiment, the artificial CSF comprises Hp.

In an embodiment, the artificial CSF comprises from about 2 μM to about 20 mM Hp. In an embodiment, the artificial CSF comprises from about 2 μM to about 5 mM Hp. In an embodiment, the artificial CSF comprises from about 100 μM to about 5 mM Hp. In an embodiment, the artificial CSF comprises from about 2 μM to about 300 μM Hp. In an embodiment, the artificial CSF comprises from about 5 μM to about 50 μM Hp. In an embodiment, the artificial CSF comprises from about 10 μM to about 30 μM Hp.

In an embodiment, the artificial CSF comprises at least an equimolar amount of Hp to the concentration of cell-free Hb in the CFS of the subject following the haemorrhage. In another embodiment, the artificial CSF comprises from about 3 μM to about 300 μM Hp.

### Adjunct Therapy

The methods of treating or preventing an adverse secondary neurological outcome in a subject following haemorrhagic stroke, as described herein, may suitably be performed together, either sequentially or in combination (e.g., at the same time), with one or more another treatment strategies designed to reduce, inhibit, prevent or otherwise alleviate one or more adverse secondary neurological outcome in a subject following haemorrhagic stroke. Thus, in an embodiment, the method further comprises administering to the subject a second agent for treating or preventing an adverse secondary neurological outcome following an intraventricular haemorrhage. Suitable other treatment strategies or second agents for treating or preventing an adverse secondary neurological outcome following an intraventricular haemorrhage will be familiar to persons skilled in the art, illustrative examples of which include:
(i) Coagulopathy correction—e.g., using vitamin K antagonists (VKAs), novel oral anticoagulants (NOAC, such as dabigatran, rivaroxaban, and apixaban), factor eight inhibitor bypass activity (FEIBA) and activated recombinant factor VII (rFVIIa), prothrombin complex concentrate, activated charcoal, antiplatelet therapy (APT), and aspirin monotherapy;
(ii) Lowering blood pressure—e.g., antihypertensive agents, illustrative examples of which include (i) diuretics, such as thiazides, including chlorthalidone, chlorthiazide, dichlorophenamide, hydroflumethiazide, indapamide, and hydrochlorothiazide; loop diuretics, such as bumetanide, ethacrynic acid, furosemide, and torsemide; potassium sparing agents, such as amiloride, and triamterene; and aldosterone antagonists, such as spironolactone, epirenone, and the like; (ii) beta-adrenergic blockers such as acebutolol, atenolol, betaxolol, bevantolol, bisoprolol, bopindolol, carteolol, carvedilol, celiprolol, esmolol, indenolol, metaprolol, nadolol, nebivolol, penbutolol, pindolol, propanolol, sotalol, tertatolol, tilisolol, and timolol, and the like; (iii) calcium channel blockers such as amlodipine, aranidipine, azelnidipine, barnidipine, benidipine, bepridil, cinaldipine, clevidipine, diltiazem, efonidipine, felodipine, gallopamil, isradipine, lacidipine, lemildipine, lercanidipine, nicardipine, nifedipine, nilvadipine, nimodepine, nisoldipine, nitrendipine, manidipine, pranidipine, and verapamil, and the like; (iv) angiotensin converting enzyme (ACE) inhibitors such as benazepril; captopril; cilazapril; delapril; enalapril; fosinopril; imidapril; losinopril; moexipril; quinapril; quinaprilat; ramipril; perindopril; perindropril; quanipril; spirapril; tenocapril; trandolapril, and zofenopril, and the like; (v) neutral endopeptidase inhibitors such as omapatrilat, cadoxatril and ecadotril, fosidotril, sampatrilat, AVE7688, ER4030, and the like; (vi) endothelin antagonists such as tezosentan, A308165, and YM62899, and the like; (vii) vasodilators such as hydralazine, clonidine, minoxidil, and nicotinyl alcohol, and the like; (viii) angiotensin II receptor antagonists such as candesartan, eprosartan, irbesartan, losartan, pratosartan, tasosartan, telmisartan, valsartan, and EXP-3137, F16828K, and RNH6270, and the like; (ix) a/s adrenergic blockers as nipradilol, arotinolol and amosulalol, and the like; (x) alpha 1 blockers, such as terazosin, urapidil, prazosin, bunazosin, trimazosin, doxazosin, naftopidil, indoramin, WHIP 164, and XENOIO, and the like; and (xi)-alpha 2 agonists such as lofexidine, tiamenidine, moxonidine, rilmenidine and guanobenz, and the like.
(ii-b) Vasodilators—e.g., hydralazine (apresoline), clonidine (catapres), minoxidil (loniten), nicotinyl alcohol (roniacol), sydnone and sodium nitroprusside.
(iii) Management of Seizures, Glucose and Temperature—e.g., antiepileptic drugs, insulin infusions to control blood glucose levels, maintenance of normo-thermia and therapeutic cooling;
(iv) Surgical treatment—e.g., hematoma evacuation (surgical clot removal), decompressive craniectomy (DC), minimally invasive surgery (MIS; such as needle aspiration of basal ganglia haemorrhages), MIS with recombinant tissue-type plasminogen activator (rtPA);
(v) Timing of Surgery—e.g., from 4 to 96 hours after symptom onset;
(vi) Thrombin Inhibition—e.g., hirudin, argatroban, serine protease inhibitors (e.g., nafamostat mesilate);
(vii) Prevention of Heme and Iron Toxicity—e.g., non-specific heme oxygenase (HO) inhibitors such as tin-mesoporphyrin, iron chelators such as deferoxamine;
(viii) PPARg antagonists and agonists—e.g., rosiglitazone, 15d-PGJ2 and pioglitazone;
(ix) Inhibition of microglial activation—e.g., tuftsin fragment 1-3 (a microglia/macrophage inhibitory factor) or minocycline (a tetracycline-class antibiotic);
(x) Upregulation of NF-Erythroid-2-Related Factor 2 (Nrf2);
(xi) Cyclo-Oxygenase (COX) Inhibition—e.g., celecoxib (a selective COX-2 inhibitor); (xii) Matrix Metalloproteinases;
(xiii) TNF-α modulators—e.g., adenosine receptor agonists such as CGS 21680, TNF-α-specific antisense oligodeoxynucleotides such as ORF4-PE; and
(xiv) Raising blood pressure—e.g., catecholamines
(xv) Inhibitors of TLR4 signalling—e.g., antibody Mts510 and TAK-242 (a cyclohexene derivative);

In an embodiment, the second agent is a vasodilator. Suitable vasodilators will be familiar to persons skilled in the art, illustrative examples of which include sydnone and sodium nitroprusside. Thus, in an embodiment disclosed herein, the second agent is selected from the group consisting of a sydnone and sodium nitroprusside.

### Artificial CSF

In another aspect disclosed herein, there is provided an artificial cerebral spinal fluid (CSF) comprising Hp, as herein described.

Artificial cerebrospinal fluid (aCSF) is typically a fluid that mimics natural CSF, including by salt content. Suitable compositions of aCSF will be familiar to persons skilled in the art, illustrative examples of which are described in US 2006/0057065 and Matzneller et al. (Pharmacology, 2016; 97(5-6):233-44, the contents of which are incorporated herein by reference in their entirety). The aCSF may comprise NaCl at a similar concentration to that found in natural CSF, as will be familiar to persons skilled in the art, and would typically include concentrations within about 15%, more preferably within about 10% of the concentration of NaCl in natural CSF. The aCSF may comprise NaHCO₃ at a similar concentration to that found in natural CSF, as will be familiar to persons skilled in the art, and would typically include concentrations within about 15%, more preferably within about 10% of the concentration of NaHCO₃ in natural CSF. The aCSF may comprise KCl at a similar concentration to that found in natural CSF, as will be familiar to persons skilled in the art, and would typically include concentrations within about 15%, more preferably within about 10% of the concentration of KCl in natural CSF. The aCSF may comprise NaH₂PO₄ at a similar concentration to that found in natural CSF, as will be familiar to persons skilled in the art, and would typically include concentrations within about 15%, more preferably within about 10% of the concentration of NaH₂PO₄ in natural CSF. The aCSF may comprise MgCl₂ at a similar concentration to that found in natural CSF, as will be familiar to persons skilled in the art, and would typically include concentrations within about 15%, more preferably within about 10% of the concentration of MgCl₂ in natural CSF. The aCSF may comprise glucose at a similar concentration to that found in natural CSF, as will be familiar to persons skilled in the art, and would typically include concentrations within about 15%, more preferably within about 10% of the concentration of glucose in natural CSF. Alternatively, the artificial CSF may omit glucose so as to reduce the likelihood of bacterial growth in any catheter used to administer the aCSF to a subject.

In an embodiment, the artificial CSF comprises from about 2 μM to about 20 mM Hp. In an embodiment, the artificial CSF comprises from about 2 μM to about 5 mM Hp. In an embodiment, the artificial CSF comprises from about 100 μM to about 5 mM Hp. In an embodiment, the artificial CSF comprises from about 2 μM to about 300 μM Hp. In an embodiment, the artificial CSF comprises from about 5 μM to about 50 μM Hp. In an embodiment, the artificial CSF comprises from about 10 μM to about 30 μM Hp.

In an embodiment, the artificial CSF comprises at least an equimolar amount of Hp to the concentration of cell-free Hb in the CFS of the subject following the haemorrhage. In another embodiment, the artificial CSF comprises from about 3 μM to about 300 μM Hp.

In an embodiment, the Hp is selected from the group consisting of an Hp1-1 homodimer, an Hp1-2 multimer, an Hp2-2 multimer and a combination of any of the foregoing. In an embodiment, the Hp comprises, consists or consists essentially of an Hp2-2 multimer.

### Pharmaceutical Compositions

In another aspect disclosed herein, there is provided a pharmaceutical composition for treating or preventing an adverse secondary neurological outcome in a subject following an intraventricular haemorrhage in accordance with the methods described herein, the composition comprising a therapeutically effective amount of Hp, as described herein, and a pharmaceutically acceptable carrier.

In another aspect disclosed herein, there is provided a pharmaceutical composition for use in treating or preventing an adverse secondary neurological outcome in a subject following an intraventricular haemorrhage in accordance with the methods described herein, the composition comprising a therapeutically effective amount of Hp, as described herein, and a pharmaceutically acceptable carrier.

In an embodiment, the composition comprises from about 2 μM to about 20 mM Hp. In an embodiment, the composition comprises from about 2 μM to about 5 mM Hp. In an embodiment, the composition comprises from about 100 μM to about 5 mM Hp, or a functional analogue thereof. In an embodiment, the composition comprises from about 2 μM to about 300 μM Hp. In an embodiment, the composition comprises from about 5 μM to about 50 μM Hp. In an embodiment, the composition comprises from about 10 μM to about 30 μM Hp.

In an embodiment, the composition comprises at least an equimolar amount of Hp to the concentration of cell-free Hb in the CFS of the subject following the haemorrhage. In another embodiment, the composition comprises from about 3 μM to about 300 μM Hp.

In an embodiment, the Hp is selected from the group consisting of an Hp1-1 homodimer, an Hp1-2 multimer, an Hp2-2 multimer and a combination of any of the foregoing. In an embodiment, the Hp comprises, consists or consists essentially of an Hp2-2 multimer.

In another aspect disclosed herein, there is provided use of a therapeutically effective amount of Hp, as described herein, in the manufacture of a medicament for treating or preventing an adverse secondary neurological outcome in a subject following an intraventricular haemorrhage in accordance with the methods described herein.

In an embodiment, the pharmaceutical compositions disclosed herein are formulated for intrathecal administration. Suitable intrathecal delivery systems will be familiar to persons skilled in the art, illustrative examples of which are described by Kilburn et al. (2013, Intrathecal Administration. In: Rudek M., Chau C., Figg W., McLeod H. (eds) Handbook of Anticancer Pharmacokinetics and Pharmacodynamics. Cancer Drug Discovery and Development. Springer, New York, N.Y.), the contents of which are incorporated herein by reference in their entirety.

In another embodiment, the pharmaceutical compositions disclosed herein are formulated for intracranial administration. Suitable intrathecal delivery systems will be familiar to persons skilled in the art, illustrative examples of which are described by Upadhyay et al. (2014, PNAS, 111(45):16071-16076), the contents of which are incorporated herein by reference in their entirety.

In another embodiment, the pharmaceutical compositions disclosed herein are formulated for intracerebroventricular administration. Suitable intrathecal delivery systems will be familiar to persons skilled in the art, illustrative examples of which are described by Cook et al. (2009, Pharmacotherapy. 29(7):832-845), the contents of which are incorporated herein by reference in their entirety.

Suitable pharmaceutical compositions and unit dosage forms thereof may comprise conventional ingredients in conventional proportions, with or without additional active compounds or principles, and such unit dosage forms may contain any suitable effective amount of the active ingredient commensurate with the intended daily dosage range to be employed.

### Kits

In another aspect disclosed herein, there is provided a kit comprising the artificial CSF, as described herein, or the pharmaceutical composition, as described herein. The active agents, as herein described (including Hp) may be presented in the form of a kit of components adapted for allowing concurrent, separate or sequential administration of the active agents. Each carrier, diluent, adjuvant and/or excipient must be “pharmaceutically acceptable” insofar as it is compatible with the other ingredients of the composition and physiologically tolerated by the subject. The compositions may conveniently be presented in unit dosage form and may be prepared by methods well known in the art of pharmacy. Such methods include the step of bringing into association the active ingredient with the carrier, which constitutes one or more accessory ingredients. In general, the compositions are prepared by uniformly and intimately bringing into association the active ingredient with liquid carriers, diluents, adjuvants and/or excipients or finely divided solid carriers or both, and then if necessary shaping the product.

The present invention therefore relates particularly to the following embodiments [1] to [74]:
[1.] A method of treating or preventing an adverse secondary neurological outcome in a subject following a haemorrhagic stroke accompanied by extravascular erythrolysis and release of cell-free haemoglobin (Hb) into a cerebral spinal fluid (CSF), the method comprising exposing the CSF of a subject in need thereof to a therapeutically effective amount of haptoglobin (Hp) and for a period of time sufficient to allow the Hp to form a complex with, and thereby neutralise, the cell-free Hb.
[2.] The method of item [1], wherein the haemorrhagic stroke is a spontaneous haemorrhage or a traumatic haemorrhage.
[3.] The method of item [1] or item [2], wherein the haemorrhagic stroke is an intraventricular haemorrhage or a subarachnoid haemorrhage.
[4.] The method of item [3], wherein the subarachnoid haemorrhage is an aneurysmal subarachnoid haemorrhage.
[5.] The method of any one of items [1] to [4], wherein the adverse secondary neurological outcome is selected from the group consisting of a delayed ischaemic neurological deficit (DIND), delayed cerebral ischaemia (DCI), neurotoxicity, inflammation, nitric oxide depletion, oxidative tissue injury, cerebral vasospasm, cerebral vasoreactivity and oedema.
[6.] The method of item [5], wherein the adverse secondary neurological outcome is a cerebral vasospasm.
[7.] The method of item [5], wherein the adverse secondary neurological outcome is a delayed ischaemic neurological deficit.
[8.] The method of item [5], wherein the adverse secondary neurological outcome is delayed cerebral ischaemia.
[9.] The method of any one of items [1] to [8], wherein the adverse secondary neurological outcome is an adverse secondary neurological outcome within the brain parenchyma.
[10.] The method of any one of items [1] to [9], comprising exposing the CSF to the Hp, or the functional analogue thereof, within about 21 days after onset of the haemorrhage.
[11.] The method of item [10], comprising exposing the CSF to the Hp from about 2 days to about 4 days after onset of the haemorrhage.
[12.] The method of item [10], comprising exposing the CSF to the Hp from about 5 days to about 14 days after onset of the haemorrhage.
[13.] The method of any one of items [1] to [12], comprising intracranially administering to the subject the Hp.
[14.] The method of any one of items [1] to [12], comprising intrathecally administering to the subject the Hp.
[15.] The method of item [14], wherein the Hp is intrathecally administering into the spinal canal.
[16.] The method of item [14], wherein the Hp is intrathecally administering into the subarachnoid space.
[17.] The method of any one of items [1] to [12], comprising intracerebroventricularly administering to the subject the therapeutically effective amount of the Hp.
[18.] The method of any one of items [13] to [17], wherein the period of time to which the CSF is exposed to the therapeutically effective amount of Hp is at least about 2 minutes.
[19.] The method of item [18], wherein the period of time to which the CSF is exposed to the therapeutically effective amount of Hp is from about 2 minutes to about 45 minutes.
[20.] The method of item [18], wherein the period of time to which the CSF is exposed to the therapeutically effective amount of Hp is from about 2 minutes to about 20 minutes.
[21.] The method of item [18], wherein the period of time to which the CSF is exposed to the therapeutically effective amount of Hp is from about 4 minutes to about 10 minutes.
[22.] The method of any one of items [1] to [21], wherein the therapeutically effective amount of Hp is at least an equimolar amount to the concentration of cell-free Hb in the CSF of the subject following the haemorrhage.
[23.] The method of item [22], further comprising (i) obtaining a CSF sample from the subject following the haemorrhage and prior to exposing the CSF to the Hp; (ii) measuring the amount of cell-free Hb in the CSF sample obtained in step (i); and (iii) determining the at least equimolar amount of Hp based on the concentration of cell-free Hb from step (ii).
[24.] The method of any one of items [1] to [23], wherein the therapeutically effective amount of Hp is from about 2 μM to about 20 mM.
[25.] The method of item [24], wherein the therapeutically effective amount of Hp is from about 2 μM to about 300 μM.
[26.] The method of item [24], wherein the therapeutically effective amount of Hp is from about 5 μM to about 50 μM.
[27.] The method of item [24], wherein the therapeutically effective amount of Hp is from about 10 μM to about 30 μM.
[28.] The method of any one of items [1] to [27], further comprising removing Hp:cell-free Hb complexes formed in the CSF.
[29.] The method of any one of items [1] to [28], comprising exposing the CSF to the therapeutically effective amount of Hp extracorporeally.
[30.] The method of item [29], comprising:
   (i) obtaining sample of CSF from the CSF compartment of the subject following the haemorrhage;
   (ii) adding to the CSF sample of step (i) Hp to obtain an Hp-enriched CSF sample;
   (iii) administering the Hp-enriched CSF sample to the subject, thereby exposing the CSF compartment of the subject to a therapeutically effective amount of Hp for a period of time sufficient to allow the Hp to form a complex with cell-free Hb in the CSF compartment of the subject; and
   (iv) optionally repeating steps (i) to (iii).
[31.] The method of item [29], comprising:
   (i) removing a volume of CSF from the CSF compartment of the subject following the haemorrhage;
   (ii) providing an artificial CSF comprising Hp;
   (iii) administering the artificial CSF of (ii) to the subject, thereby exposing the CSF compartment of the subject to a therapeutically effective amount of Hp for a period of time sufficient to allow the Hp to form a complex with cell-free Hb in the CSF compartment of the subject; and
   (iv) optionally repeating steps (i) to (iii).
[32.] The method of item [29], comprising:
   (i) obtaining CSF from the subject following the haemorrhage;
   (ii) exposing the CSF from step (i) to the Hp under conditions to allow the Hp, or the functional analogue thereof, to form a complex with cell-free Hb in the CSF;
   (iii) extracting the Hp:cell-free Hb complexes from the CSF following step (ii) to obtain an Hb-diminished CSF that has a lower amount of cell-free Hb when compared to the CSF from step (i);
   (iv) optionally repeating steps (ii) and (iii) to obtain an Hb-diminished CSF that is substantially free of cell-free Hb; and
   (v) administering the Hb-diminished CSF obtained from step (iii) or step (iv) to the CSF compartment of the subject.
[33.] The method of item [32], further comprising adding to the Hb-diminished CSF prior to step (vi) a therapeutically effective amount of Hp.
[34.] The method of item [32] or item [33], wherein the Hp is immobilised on a substrate.
[35.] The method of item [34], wherein the substrate is an affinity chromatography resin.
[36.] The method of item [35], wherein step (ii) comprises passing the CSF from step (i) through an affinity chromatography resin under conditions that allow the cell-free Hb in the CSF to bind to the resin; wherein step (iii) comprises eluting the CSF from the resin following step (ii); and wherein step (iv) comprising recovering the eluted CSF.
[37.] The method of any one of items [32] to [36], optionally comprising washing the CSF compartment following step (i) with a wash solution.
[38.] The method of item [37], wherein the wash solution is an artificial CSF.
[39.] The method of item [31] or item [38], wherein the artificial CSF comprises NaCl, KCl, KH₂PO₄, NaHCO₃, MgCl₆H₂O, CaCl₂H₂O and glucose.
[40.] The method of any one of items [37] to [39], wherein the wash solution comprises Hp.
[41.] The method of item [40], wherein the wash solution comprises from about 2 μM to about 20 mM Hp.
[42.] The method of item [41], wherein the wash solution comprises from about 2 μM to about 300 μM Hp.
[43.] The method of any one of items [1] to [42], comprising:
   (i) removing the CSF from the subject following the haemorrhage;
   (ii) rinsing the CSF compartment of the subject following step (i) with a wash solution comprising a therapeutically effective amount of Hp;
   (iii) optionally repeating step (ii); and
   (iv) administering to the CSF compartment of the subject, following step (ii) or step (iii), an artificial CSF.
[44.] The method of item [43], wherein the artificial CSF comprises NaCl, KCl, KH₂PO₄, NaHCO₃, MgCl₆H₂O, CaCl₂H₂O and glucose.
[45.] The method of item [43] or item [44], wherein the artificial CSF comprises Hp.
[46.] The method of item [45], wherein the artificial CSF comprises from about 2 μM to about 20 mM Hp.
[47.] The method of item [46], wherein the artificial CSF comprises from about 2 μM to about 300 μM Hp.
[48.] The method of any one of items [1] to [47], wherein the Hp is selected from the group consisting of an Hp1-1 homodimer, an Hp1-2 multimer, an Hp2-2 multimer and a combination of any of the foregoing.
[49.] The method of item [48], wherein the Hp comprises an Hp2-2 multimer.
[50.] The method of any one of items [1] to [49], wherein the Hp is a recombinant protein.
[51.] The method of any one of items [1] to [49], wherein the Hp is plasma derived.
[52.] The method of any one of items [1] to [51], further comprising administering to the subject a second agent for treating or preventing an adverse secondary neurological outcome following an intraventricular haemorrhage.
[53.] The method of item [52], wherein the second agent is a vasodilator.
[54.] The method of item [52] or item [53], wherein the second agent is selected from the group consisting of a sydnone and sodium nitroprusside.
[55.] An artificial cerebral spinal fluid (CSF) comprising Hp.
[56.] The artificial CSF of item [55], wherein the Hp is present in an amount of from about 2 μM to about 20 mM.
[57.] The artificial CSF of item [55], wherein the Hp is present in an amount of from about 5 μM to about 300 μM.
[58.] The artificial CSF of item [55], wherein the Hp is present in an amount of from about 5 μM to about 50 μM.
[59.] The artificial CSF of item [58], wherein the Hp is present in an amount of from about 10 μM to about 30 μM.
[60.] The artificial CSF of any one of items [57] to [59], wherein the Hp is selected from the group consisting of an Hp1-1 homodimer, an Hp1-2 multimer, an Hp2-2 multimer and a combination of any of the foregoing.
[61.] The artificial CSF of item [60], comprising an Hp2-2 multimer.
[62.] The artificial CSF of item [59] or [60], wherein the Hp is a recombinant protein.
[63.] The artificial CSF of item [57] or [58], wherein the Hp is plasma derived.
[64.] A pharmaceutical composition for treating or preventing an adverse secondary neurological outcome in a subject following a haemorrhagic stroke in accordance with the method of any one of items [1] to [54], the composition comprising a therapeutically effective amount of Hp, and a pharmaceutically acceptable carrier.
[65.] A pharmaceutical composition for use in treating or preventing an adverse secondary neurological outcome in a subject following a haemorrhagic stroke in accordance with the method of any one of items [1] to [54], the composition comprising a therapeutically effective amount of Hp, and a pharmaceutically acceptable carrier.
[66.] The composition of item [64] or item [65], wherein the Hp is present in an amount of from about 2 μM to about 20 mM.
[67.] The composition of item [66], wherein the Hp is present in an amount of from about 2 μM to about 300 μM.
[68.] The composition of item [66], wherein the Hp is present in an amount of from about 5 μM to about 50 μM.
[69.] The composition item [66], wherein the Hp is present in an amount of from about 10 μM to about 30 μM.
[70.] The composition of any one of items [64] to [69], wherein the Hp is selected from the group consisting of an Hp1-1 homodimer, an Hp1-2 multimer, an Hp2-2 multimer and a combination of any of the foregoing.
[71.] The composition of item [70], comprising a Hp2-2 multimer. According to a further embodiment, the composition of item [70] comprises a Hp1-1 homodimer.
[72.] Use of a therapeutically effective amount of haptoglobin (Hp) in the manufacture of a medicament for treating or preventing an adverse secondary neurological outcome in a subject following a haemorrhagic stroke in accordance with the method of any one of items [1] to [54].
[73.] A therapeutically effective amount of haptoglobin (Hp) for use in the treatment or prevention of an adverse secondary neurological outcome in a subject following a haemorrhagic stroke in accordance with the method of any one of items [1] to [54].
[74.] A kit comprising the artificial CSF of any one of items [55] to [63] or the composition of any one of items [64] to [71].

Those skilled in the art will appreciate that the invention described herein is susceptible to variations and modifications other than those specifically described. It is to be understood that the invention includes all such variations and modifications which fall within the spirit and scope. The invention also includes all of the steps, features, compositions and compounds referred to or indicated in this specification, individually or collectively, and any and all combinations of any two or more of said steps or features.

Certain embodiments of the invention will now be described with reference to the following examples which are intended for the purpose of illustration only and are not intended to limit the scope of the generality hereinbefore described.

### Examples

### Materials and Methods

### A. Ex Vivo Vascular Function Experiments

### Isolation of Target Vessels

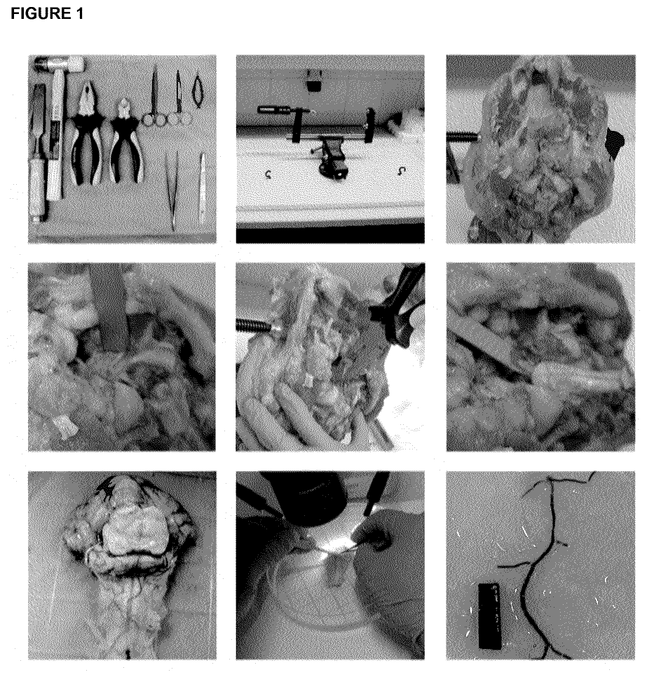

Basilar arteries were isolated from fresh heads of slaughtered pigs from the local slaughterhouse (SBZ, Zurich, Switzerland). Heads were positioned supine in a customized holding device and the clivus was exposed from the level of the occipital condyles up to the posterior nasal aperture by resection of residual soft tissue. The dura mater was carefully detached from the anterior rim of the foramen magnum and slightly mobilized to create space for the craniotomy. Bilateral paramedian osteotomy of the clivus and partial condylectomy using a chisel lead to exposure of the ventral dura mater. Dura mater was carefully removed leaving the perivascular arachnoid cisterns of the ventral brainstem intact. Cranial nerves III-XII were dissected to mobilize the brainstem. The brainstem was isolated by sharp dissection at the level of the pontomesencephalic junction and the cerebellar peduncles and transferred to pre-cooled (4° C.) buffer solution. Using a dissection microscope, the vertebral arteries were identified as an anatomical landmark and cut 2 mm proximal to the vertebrobasilar junction. Careful arachnoidal preparation allowed stepwise mobilisation of the basilar artery avoiding excessive mechanical manipulation of the vessel. The basilar artery segment between the vertebrobasilar junction and the caudal cerebral artery were used to prepare up to 6 vascular rings (length of 2 mm per ring) (FIG. 1).

During all surgical preparations special attention was paid to avoid any compression or distension of the arteries. The time between the death of the animal and the transfer of the dissected vessels into the buffer was kept below 90 minutes.

### Preparation of Buffers and Chemicals

Krebs-Henseleit-Buffer (KHB) was prepared in batches of five liters. Stock Solutions of 2.27 M NaCl and KCl as well as 1.00 M KH₂PO₄ were prepared. Following the right order of chemicals and thorough mixing after each addition step are crucial for the preparation of this buffer, since ionic composition of several components is near to the maximum solubility; especially for calcium hydrogen carbonate which tends to precipitate as lime (CaCO₃) but also calcium phosphate and gypsum (CaSO₄). In 4 liters of distilled water at room temperature, 10.35 ml of 2.27 M KCl and 138.1 ml of 2.27 M NaCl were mixed. 10.50 g NaHCO₃ are added and the buffer was agitated until all salts were completely dissolved. 6.0 ml of 1.00 M KH₂PO₄ were added and the solution was again agitated. 1.84 g of CaCl₂×2 H₂O followed by 1.48 g MgSO₄×7 H₂O were dissolved in the buffer. 121 ml of 2.27 M NaCl were added. The buffer was heated to exactly 37° C. and equilibrated by bubbling with 5% CO₂ and 95% O₂ for at least one hour. pH was adjusted by adding concentrated ortho-phosphoric acid dropwise to 7.40. Volume was adjusted with distilled water to 5 liters. The buffer was stored at room temperature (the salts precipitate at 4° C.). Glucose was added only immediately before use to a concentration of 2 g/I to prevent premature microbiological deterioration of the buffer. Final ionic composition for KHB is: 143 mM Na⁺, 5.90 mM K⁺, 1.20 mM Mg₂⁺, 2.50 mM Ca₂⁺, 125 mM Cl⁻, 25.0 mM HCO₃⁻, 1.20 mM of SO₄²⁺, 1.20 mM of PO₄³⁻, and 11.1 mM Glucose.

### MAHMA-NONOate

MAHMA-NONOate (ENZO Life Sciences, Lausen, Switzerland) was dissolved in 20 mM NaOH to a stock concentration of 10 mM and stored in small aliquots at −80° C. Prior to usage, the chemical was diluted to a concentration of 25 μM in 5 mM NaOH and stored on ice. It is crucial to keep MAHMA-NONOate at high pH immediately until it is used, since at physiological pH the half-life of this chemical is only in the range of seconds to minutes, depending on the temperature.

### PGF2α

Prostaglandin F2a (PGF2α) Sigma, Buchs, Switzerland) was dissolved in PBS pH 7.4 to a stock concentration of 10 mM and stored at −80° C. in small aliquots until usage.

CSF Samples of aSAH Patients

Consecutive patients admitted to our Neurocritical Care Unit, University Hospital of Zurich (an academic tertiary care center), with diagnosis aSAH and insertion of an EVD due to hydrocephalus were screened for study inclusion between April 2017 and December 2018. The study was approved by the local ethical review board and written consent was obtained from all patients or their legal representatives before study inclusion.

Exclusion criteria were defined as follows: unknown source of bleeding within 72 h, failure to secure aneurysm within 72 hours, rebleeding from unsecured aneurysms and age <18 and >80 years.

After aneurysm repair, ventricular CSF from the EVD catheter was sampled daily between day 0 (day of bleeding event) and day 14. CSF was centrifuged at 1500 G for 15 minutes (Capricorn CEP 2000 Benchtop centrifuge, Capricorn labs, UK). Supernatant was collected without further dilution for spectrophotometry.

Spectra in the visual range of liquor supernatants were recorded between 350 and 650 nm in 2 nm resolution on a Shimadzu UV-1800 spectrophotometer (Shimadzu, Japan). Spectra were deconvoluted by fitting reference spectra of known concentrations of oxy-hemoglobin (Fe²⁺), met-hemoglobin (Fe³⁺) and bilirubin by the Lawson-Hanson implementation of the non-negative least squares algorithm” with R statistical software version 4.2.3 (www.r-project.org) as described previously¹². In samples with an extinction of more than 2.0 for the soret peak, deconvolution was limited to wavelengths larger than 435 nm to compensate for the nonlinearity of the spectrophotometer at high absorptions.

CSF samples for wire myography experiments were defined as pre-haemolytic (day 1-3) and haemolytic (day 4-14). Due to limited available CSF volumes from single days, samples of consecutive days were pooled if needed.

### Wire Myography

Vascular rings were mounted on two 0.2 mm diameter pins of a Multi-Channel Myograph System 620M (Danish Myo Technology, Aarhus, Denmark) immersed in temperature controlled (37° C.) and continuously aerated (95% O₂ and 5% CO₂ gas mixture) organ baths containing 5 ml of Krebs-Henseleit-Buffer (KHB). For experiments with CSF, organ bath were equipped with 3D printed customized inlays (volume of 2.5 ml) and CSF samples diluted 1:1 with artificial CSF to reduce the needed volumes of patient samples for single experiments.

The vessels were gradually stretched to the optimal IC1/IC100 ratio determined in the previous experiments. As a pre-contracting agent prostaglandin F2a (PGF2α) was used at a 10 μM concentration. The NO mediated vasodilatory responses were induced by addition of MAHMA-NONOate to the immersion buffer. All data were recorded using Lab Chart software version 7.2.1 (AD Instruments, Hastings, UK). If not otherwise stated, the recorded hemoglobin-induced vascular function responses were normalized relative to maximum NO-dilatation without Hb exposure (=100%) and the level of tonic contraction before addition of MAHMA-NONOate (=0%), respectively, during a single experiment. The responses in the plots are therefore indicated as relative contraction.

B. Sheep Model of oxyHb Induced Vasospasms

### Animals, Housing and Care

The study was conducted according to the Swiss legal requirements for animal protection and welfare (TschG 455) and received ethical approval by the federal veterinary authorities ‘Kantonale Tierversuchskommission Zurich’ (permission No ZH234/17).

Swiss alpine female sheep (Staffelegghof, Kuttigen bei Aarau, Switzerland) with an age of 2-4 years were transferred to the experimental unit of the Veterinary Hospital of Zurich and allowed to acclimatize to the new environment for at least 7 days. Standardized screening with physical examination and blood testing was performed through a veterinarian during the acclimatization period. Animals were preoperatively fasted with free access to water for 18-24 hours.

### Anesthesia

30 minutes before induction of anesthesia, the animals' physical status was checked again by the attending veterinary anesthesiologist. Each animal was then premedicated with buprenorphine (0.01 mg/kg BW im) and medetomidine (0.07-0.1 mg/kg BW im). After 20 minutes, the sedated animal was transported to the angiography suite, the degree of sedation and the health status were again clinically assessed.

A 14 G, 3.5 inch intravenous catheter was inserted under sterile preconditioning into the left jugular vein and surgically fixed to the skin. Anesthetic state was intravenously induced by fixed dose injection of midazolam (0.1 mg/kg BWT), ketamine (3 mg/kg BWT) and variable dose injection of propofol (0.3-1.5 mg/kg BWT) to effect.

Thereafter, the larynx of each animal was desensitized using lidocaine 10% sprayed directly and under visual control through a laryngoscope. After allowance of 20-30 seconds, the animals tracheas were then intubated using 11 or 12 mm ID-sized appropriately long silicone endotracheal tubes. The animals were then positioned in left lateral recumbency onto a flexible vacuum mattress on the examination table of the Allura Clarity angiography suite.

They were immediately connected to a coaxial circle breathing system of appropriate size and artificial ventilation was started at a rate of 6-8 breaths per minute and 10 mL/kg BWT of tidal volume. Isoflurane was delivered in Oxygen at 0.8-2 Vol % throughout the whole procedure. A pulse oximeter probe was connected to the animal to assess and ensure adequate hemoglobin oxygenation status throughout the whole procedure. Ventilation was adjusted to maintain normocapnia as assessed by capnography (end-tidal CO₂) and by arterial blood gas measurement as well as by calculation the arterial-to-alveolar difference and using Bohr's alveolar gas equation and thereby estimating the relative functioning of the ventilation-perfusion match. Maintenance of normal and constant arterial partial pressure of CO₂ (2.5-5.5 kPa) was of crucial importance, as arterial CO₂ is known to represent a major factor of cerebrovascular reactivity, particularly in the range of 4-9 kPa.

A urinary catheter (Foley, Size 9), was inserted into the urinary bladder to allow urine to deflow during the procedure and for monitoring of urine production over time. Under ultrasonographic control, a 8F intravascular cannula was inserted into the right external carotid artery as a port for later introduction of intravascular catheters. Once placed, 100 IU/kg/BWT of unfractionated heparin were administered intravenously to assure anticoagulation. This was repeated every 6 hours throughout the procedure. The animals were then positioned in sternal recumbency with their front legs flexed under the neck and their hind legs flexed forward as a final body position that was maintained for the rest of the procedure unvaried.

Both auricular arteries were cannulated using 20G Surflo Terumo® catheters for continuous direct arterial blood pressure monitoring and arterial blood sampling for intermittent blood gas analysis.

Animals were instrumented for continuous measurement (Datex-Ohmeda S3 compact life signs monitor) and recording (via a laptop computer) of heart rate, electrocardiogram, invasive direct arterial blood pressure, oxygen hemoglobin saturation, and inspiratory and expiratory concentrations of oxygen, carbon dioxide and isoflurane. Animals were administered lactated Ringer's solution intravenously over the whole duration of the procedure at a standard rate of 3 mL/kg BWT/hr, adjusted when needed to maintain normotension (60-100 mmHg mean arterial blood pressure) and normal urine production of 2 mL/kg/hr.

The anesthetic state was maintained and when necessary anesthesia depths varied by adjusting isoflurane inspired concentration and/or a propofol variable rate infusion administered intravenously by means of a syringe pump (Perfusor®, BBraun; rate 0.5-2 mg/kg/hr).

Furthermore, to reduce movement artifacts and to facilitate artificial respiration, rocuronium was administered intermittently at a dose of 0.5 mcg/kg intravenously every 2 hours or when clinical assessment of the degree of muscle relaxation indicated the latter to be insufficient throughout the duration of the procedure.

At the completion of the study period, i.e., when all planned data acquisition was terminated, the animals were euthanized under anesthesia by intravenous administration of pentobarbital at 150 mg/kg. Death was confirmed by means of the attached monitoring instruments as well as by transthoracic auscultation.

### Surgical Model

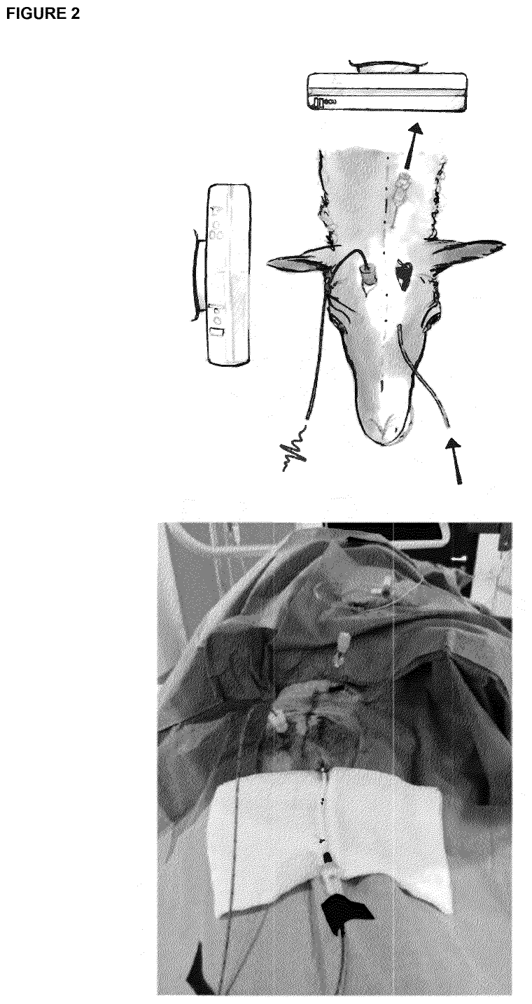

Anesthetized sheep were positioned prone in the vacuum mattress with rigid fixation of the head in a customized holding device. After clipping and disinfection of the skin, sterile draping was placed around the surgical field. A neuromonitoring probe (Luciole Medicale AG, Zurich, Switzerland) was inserted through a right frontal paramedian burr hole using a neurosurgical bolt kit (Raumedic, Helmbrechts, Germany). An external ventricular drain (EVD) (DePuys Synthes, Oberdorf, Switzerland) was inserted to the frontal horn of the left lateral ventricle through an 11 mm burr hole. A suboccipital cisternal puncture for CSF release and sampling with a standard 20G spinal needle (Dalhausen, Köln, Germany) was performed under fluoroscopic guidance. CSF samples were immediately centrifuged at 1500 G for 15 minutes. Supernatant was collected for measurement of Hb concentrations and vascular function experiments. For controlled ventricular injections, a PHD Ultra syringe pump (Harvard Apparatus, Holliston, USA) was connected to the EVD. Ventricular injections were performed with maximal flow rates of 30 ml/h. For illustration of experimental setup (see FIG. 2).

### Monitoring

Continuous monitoring of intracranial pressure (ICP), brain temperature, heart rate, blood pressure, oxygen saturation and end-tidal CO₂ was performed in all animals. Additionally, arterial blood gas analyses were analyzed every 30 minutes during the whole experimental procedure and urinary output was monitored.

### Digital Subtraction Angiography (DSA)

Angiograms were performed in an Allura Clarity angiography suite (Philips, Hamburg, Germany). The largest anastomosis between the right A. maxillaris and the extradural rete mirabile was selectively catheterized with an angiographic microcatheter through the arterial port in the right carotid artery. Biplanar oblique lateral and dorsoventral projections were acquired simultaneously. A contrast bolus of 11 ml ioversol 300 mg iodine/ml (Optiray 300, Guebert AG, Zurich, Switzerland) was injected with 2 ml/s through the microcatheter with a high pressure contrast agent injector (Accutron MR, Medtron AG, Saarbrucken, Germany).

Quantification of Vessel Diameters from DSA

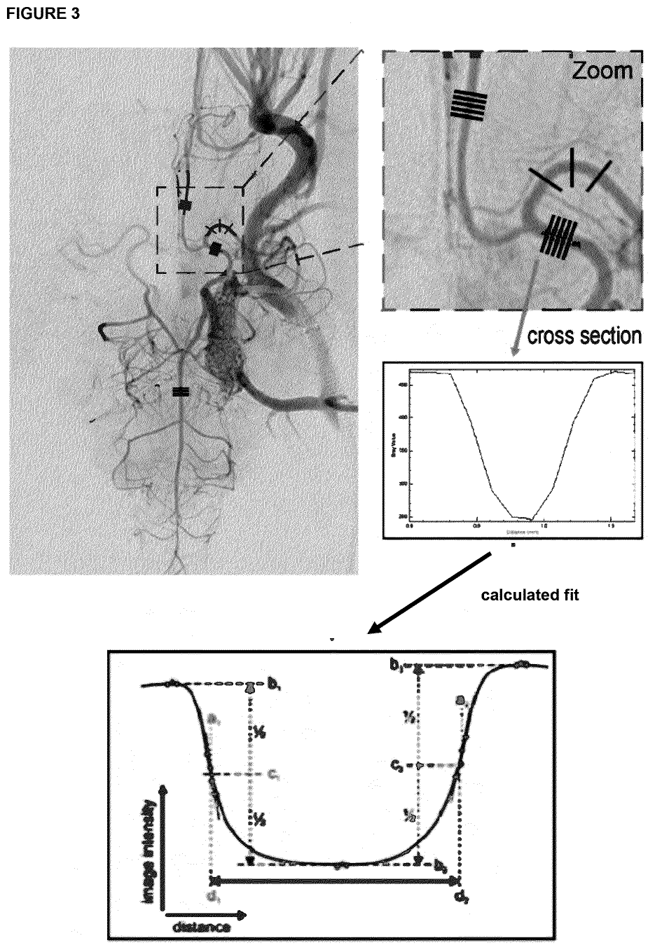

Angiography images were processed using ImageJ and vessel diameters measured with a plug-in for ImageJ¹³. For each sheep, angiograms at the timepoints pre-aCSF, pre-treatment and post-treatment (60 minutes after Hb or HbHp infusion) were compared. Within the image sequence of the single DSA at the mentioned time points, lateral projections served to define the beginning of the venous phase by contrast depiction of cortical vein influx to the superior sagittal sinus (“venous T sign”). For each time point a stack of the last three images of the arterial phase was intensity averaged. All vessel measurements were performed on dorsoventral projections in a stack combining the intensity averaged arterial phase of pre-aCSF, pre-treatment and post-treatment angiogram (FIG. 3). Measurements were performed in the following four vessels of the circle of Willis: Anterior cerebral artery (ACA), middle cerebral artery (MCA), cisternal part of internal carotid artery (ICA) and basilar artery (BA). For each vessel, the most proximal non-superimposed segment was selected on pre-aCSF images. In the vessels with anatomically straight course (ACA, ICA and BA) five linear regions of interest (ROI's) were automatically generated at 0.5 mm intervals. Since there is a risk of measurement errors with motion artefacts in non-perpendicular arrangement of the ROI's in the curvilinear MCA, three ROIs were defined manually. For each linear ROI vessel diameter was determined using the ImageJ plug-in¹³. The five measurements per ROI were averaged and the mean value of diameter change was calculated. Vessel-specific mean diameter changes were compared between pre-aCSF, pre-treatment and post-treatment angiography for each sheep and set in relation to treatment modality (Hb vs. Hb:Hp) using a Mann-Whitney-U and Kruskal-Wallis test.

### Magnetic Resonance Imaging (MRI)

In vivo magnetic resonance imaging was performed in a clinical 3 Tesla MRI unit (Philips ingenia, Amsterdam, Netherlands). Axial and sagittal T2-weighted images were acquired for anatomical orientation and post-surgical control of catheter placement. Dynamic 3D T1-weighted imaging was performed with high temporal resolution over 10 minutes covering the time of MnHb infusion. Dynamic 3D T1-weighted imaging was continued for the following 80 minutes in 10 minutes intervals with high spatial resolution. DICOM data was processed using HOROS software (Nimble Co LLC d/b/a Purview, Annapolis, Md. USA) for image reconstruction and analysis.

### Hemoglobin

Purified oxyhemoglobin (oxyHb, Fe²⁺) was produced from outdated blood via tangential flow filtration (TFF) as described previously¹⁴. Hb concentrations are always expressed as heme-equivalents throughout the manuscript.

### Protein Labelling for Histological Visualization

For histological analysis, purified protein solutions (Hb, Hp) were labeled with TCO-NHS-ester (Jena Bioscience, Jena, Germany). The TCO-NHS-ester was added dropwise to the purified protein solution (20 mg/mL in 100 mM NaHCO₃ buffer). After incubation for one hour at room temperature, the reaction was stopped by addition of 10% 1M Tris-HCl buffer (pH 8.0) followed by centrifugation for 30 minutes at 4000 g to remove denatured proteins. Subsequently excess reagents were removed using disposable desalting columns (PD-10 Desalting Columns, GE Healthcare, Chicago, Ill.). If needed, the labeled protein solutions were concentrated with ultrafiltration units (Amicon Ultra 15, 10 kDa NMWL, Merck Millipore, Billerica, Mass.). After processing all protein solutions were sterile filtered through a polyethersulfone membrane with 0.22 μm pore size (Steriflip filters, Merck Millipore, Burlington, Mass.) and stored at −80° C. until used.

Artificial CSF (aCSF)

The final composition of aCSF was 127 mM NaCl, 1.0 mM KCl, 1.2 mM KH₂PO₄, 26 mM NaHCO₃, 1.3 mM MgCl₂*6H₂O, 2.4 mM CaCl₂*2H₂O and 6.7 mM glucose.

### Haptoglobin and Hemoglobin-Haptoglobin Complexes

Purified Haptoglobin from human plasma (predominant phenotype 2-2) was obtained from CSL Behring AG (Bern, Switzerland). Haptoglobin from human plasma having phenotype 1-1 was also obtained from CSL Behring AG (Bern, Switzerland). Haptoglobin from human plasma having predominant phenotype 2-2 has been purified as previously described in WO 2014/055552 A1. Hb binding capacity of the Hp was quantified with HPLC. After complex formation the purity of the complex and the absence of free Hb was verified using HPLC.

### Perfusion Cell Culture for Providing Recombinant Haptoglobin 1-1

The cell culture process consists of vial thaw, cell expansion, inoculation of the perfusion reactor, a cell expansion phase in the reactor, followed by a production phase.

The cell culture supernatant is continuously drawn from the reactor during the production phase as a perfusion harvest via a cell separator and replaced by fresh medium. The perfusion harvest is collected during the reactor run. Each of the perfusion harvests is subjected to further midstream processing.

The CHO cell clone expressing the human wild type haptoglobin phenotype 1 including a HIS tag was thawed and cultivated in a seed train starting with a T80 flask (37° C., 5% C02) and elongating the cultivation to three 2 L shake Flasks. (Parameters: 37° C., 120 rpm, 80% humidity, 5% C02). A 20 L Glass Vessel of the Sartorius DCU System equipped with a 50 L BioSep device was inoculated with 3 L seedtrain. Up to day three a batch cultivation was performed, after that the perfusion was started with VVD (Vessel Volume exchanges per day) 1.0. Every second day a harvest of ca. 40 L was generated and cell degradation residues were separated by a Pall® Kleenpak™ Nova NP7 Filter Capsule 0.2 μm within the harvest line. The cell concentration (ca. 40×10⁵ cells/mL) during fermentation was adjusted to maintain a minimum glutamine concentration of 2 mM. (Fermentation Parameters: 37° C., pH 7.0, 30% pO2, 150 rpm, VVD 1.0) The Media used during the whole process was modified ProCHO5 w/8 mM L-Gln, 0.25% Pluro (Fa. Lonza). In total 22 Harvests (ca. 900 L) were generated.

Purification of rHaptoglobin1-1-His (rHp1-1)

Cell-free harvest was concentrated 30-fold using a TFF system (Pall Centramate 500 S, Pall Corporation, New York, USA) with a 30 kDa cut-off membrane. The concentrate of rHaptoglobin 1-1-HIS was loaded overnight on a NiSepharose excel column (GE Healthcare 17-3712-02) pre-equilibrated with 20 mM sodium phosphate+500 mmol/L NaCl, pH 7.4. After washing the column with equilibration buffer, rHaptoglobin1-1-His was eluted with elution buffer (20 mM sodium phosphate+500 mmol/L NaCl+150 mmol/L Imidazol, pH 7.4). The eluate was concentrated 10-fold using TFF system (Pall) with a 30 kDa cut-off membrane. To separate the rHaptoglobin1-1-His from unwanted impurities the material was loaded on a Superdex 200 pg column (GE Healthcare, Munich, Germany) pre-equilibrated with PBS pH 7.4 and the peak fractions containing the rHaptoglobin1-1-His were pooled and again concentrated using a stirred Ultrafiltration Cell (Model 402) with a UF-PES-20 membrane (Fa. Hoechst #FP08085) to a final concentration of around 40 mg/mL rHaptoglobin 1-1-HIS.

### Hn-Hb and Mn-Hb:Hp Complexes

Mn(III) Protoporphyrin IX chloride (MnPP) was inserted into the empty “heme-pocket” of tetrameric apohemoglobin, or apohemoglobin:haptoglobin complexes under alkaline conditions (NaOH 100 mM). Then the buffer was exchanged to saline. The compounds were stored at 4° C. immediately or stored in anti-freeze solution (Propylene Glycol, Glycerin, PBS 0.1M and ddH₂O in a 1:1:1:1 ratio) at −20° C. until further processing.

### C. Histological Staining and Imaging

After euthanasia of the sheep, the CSF space was flushed with 10 mL PFA 4% through the EVD with an infusion rate of 30 mL/h before harvesting of the brain. Whole brain was cut to 1 cm thick coronal slices and fixed in 4% PFA at 4° C. overnight. Then the slices were cropped to a suitable size for further processing, embedded in 4% Agarose in PBS and cut to 120 μm floating sections using a vibratome (Leica VT1000 S Vibrating blade microtome, Leica Biosystems, Wetzlar, Germany). The sections were either processed In a first step floating sections were preconditioned in permeabilization buffer (2% BSA with 0.5% Triton-X-100 in PBS) for 4 hours at room temperature followed by incubation in 2 mL blocking buffer (2% BSA in PBS) containing 40 nM Tetrazine-Cy5 (Jena Bioscience, Jena, Germany) and a FITC-coupled monoclonal antibody against α-smooth muscle actin (1:10′000, clone 1A4, Sigma) at 4° C. overnight. Then nuclei were stained with Hoechst 33342 (1:2′000 dilution, Invitrogen, Carlsbad, Calif.) for 40 minutes at room temperature. After three times washing with PBS for 15 minutes the sections were mounted with FluoroSafe (Merck Millipore, Burlington, Mass.).

Whole slide scans of the histological sections were produced by stitching together single images with a 10× magnification obtained with a Zeiss Observer.Z1 microscope coupled to a Colibri.2, an ApoTome.2 system and a motorized stage (Carl Zeiss AG, Feldbach, Switzerland). Images with 63× magnifications were obtained using a Leica SP8 confocal microscope (Leica Microsystems, Wetzlar, Deutschland).

### D. HPLC Measurements

For qualitative and quantitative size-exclusion chromatography (HPLC) samples containing Hb and or Hp were separated on an analytical BioSep-SEC-s3000 (600×7.8 mm) LC column coupled with a BioSep-SEC-s3000 (75×7.8 mm) Guard Column (Phenomenex, Torrance, Calif.) and attached to a LKB 2150 HPLC Pump (LKB-Produkter AB, Bromma, Sweden) and a Jasco UV-970 Intelligent UV/VIS Detector (JASCO International Co., Ltd., Tokyo, Japan). Potassium phosphate 20 mM pH 6.8 was used as mobile phase. The absorption was measured at 414 nm and recorded using Lab Chart software version 7.2.1 (AD Instruments, Hastings, UK). HPLC curves were analyzed and visualized with R statistical software version 4.2.3 (www.r-proectorg).

### Results

### A. Haptoglobin Restores NO-Mediated Vasodilation in Hb Exposed Porcine Basilar Arteries Ex Vivo

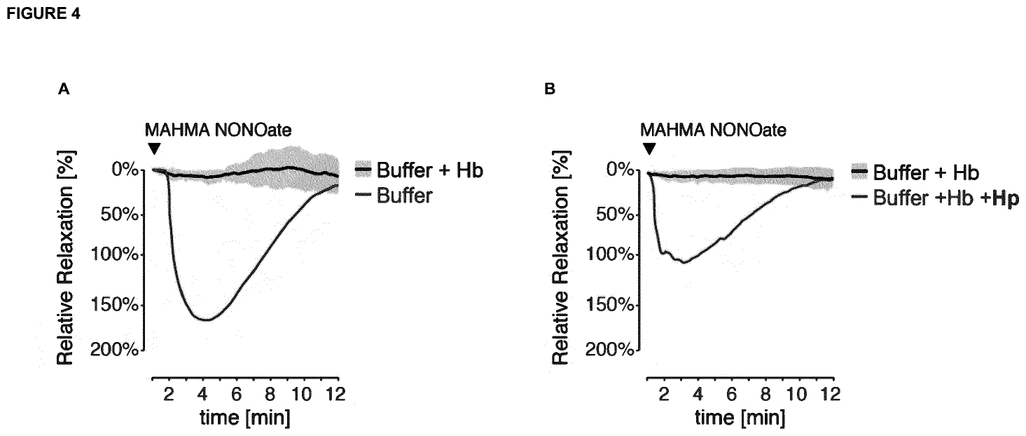

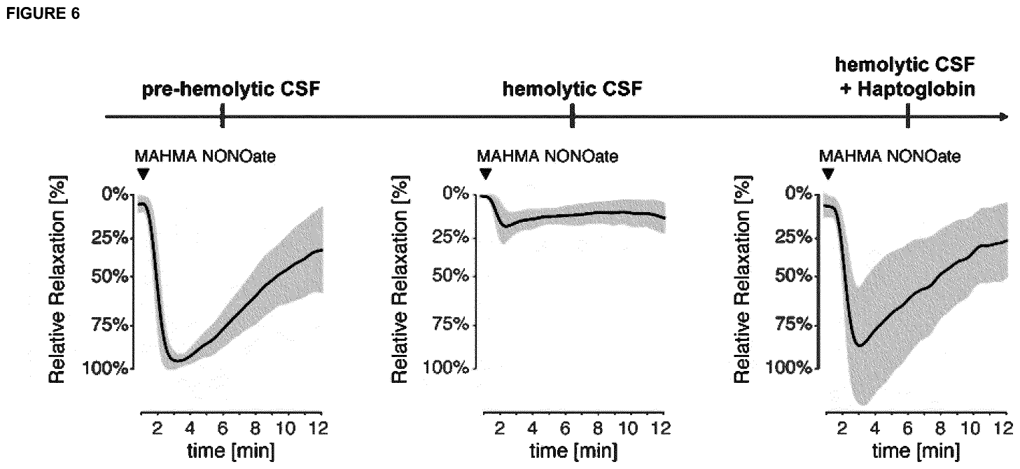

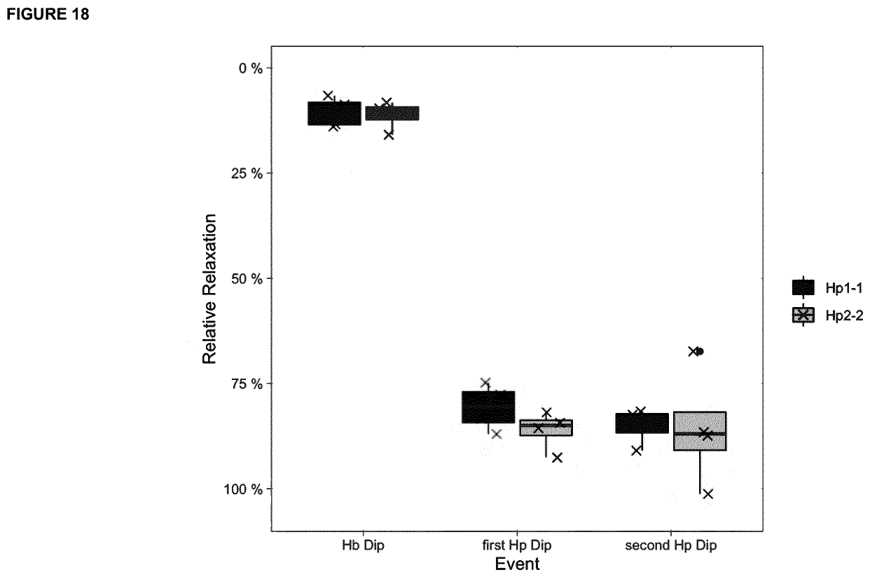

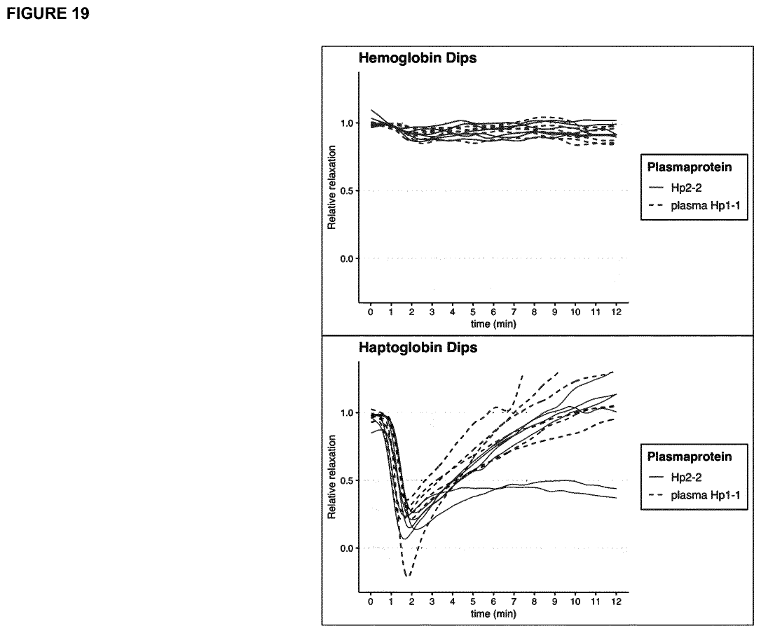

Cell-free Hb added to the Krebs-Henseleit buffer in a concentration of 10 μM completely blunted NO-induced vasodilation in wire myography experiments (FIG. 4A). Addition of equimolar Hp largely restores NO-response after an incubation period of 20 minutes (FIG. 4B). Hp also restored the vasodilatory response to exogenous NO to the extent that was observed with CSF sampled from the same patients at 2 day after bleeding, before erythrolysis occurred (FIG. 6). In vascular function experiments, no significant difference in restoration of the NO-response between plasma-derived Hp 2-2 and recombinant (FIG. 18) or plasma derived Hp 1-1 (FIG. 19) was observed.

B. Cell-Free Hb is the Major Disruptor of Arterial Nitric Oxide-Signaling in the Cerebrospinal Fluid of Patients with aSAH

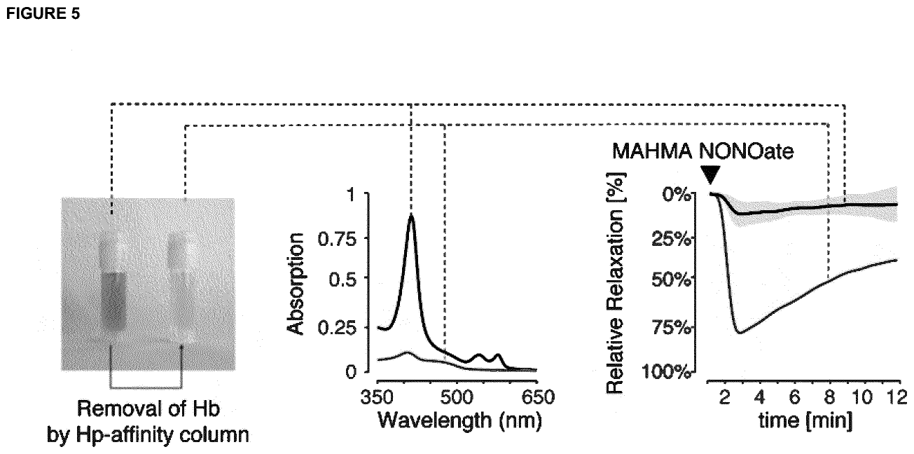

An early clinical feature of DIND is the occurrence of vasospasms in cerebral arteries. Reactions of cell-free oxyHb(Fe²⁺O₂)/deoxyHb(Fe²⁺) with the vasodilator nitric oxide (NO) may shift the balance towards vasoconstrictive forces. To investigate this mechanism of deregulation, the NO-mediated vasodilatory responses of porcine basilar arteries that were immersed in human CSF samples were quantified ex vivo. In CSF from a patient with DIND containing high cell-free Hb, the expected dilatory response to administration of a short-lived NO-donor (MAHMA-NONOate) was suppressed (FIG. 5). However, when cell-free Hb was selectively removed from the CSF with a Hp-affinity column, the physiological vasodilatory response was restored. An LC-MSMS analysis of the patient CSF before and after Hb removal demonstrated that the bulk of protein composition in the erythrolytic CSF remained unchanged. This experiment identified cell-free Hb as the major disruptor of arterial NO-signaling in the cerebrospinal fluid of patients with aSAH.

C. Hb and Hb:Hp Injected into the Lateral Ventricle Rapidly Distributes into the Subarachnoid Space In Vivo

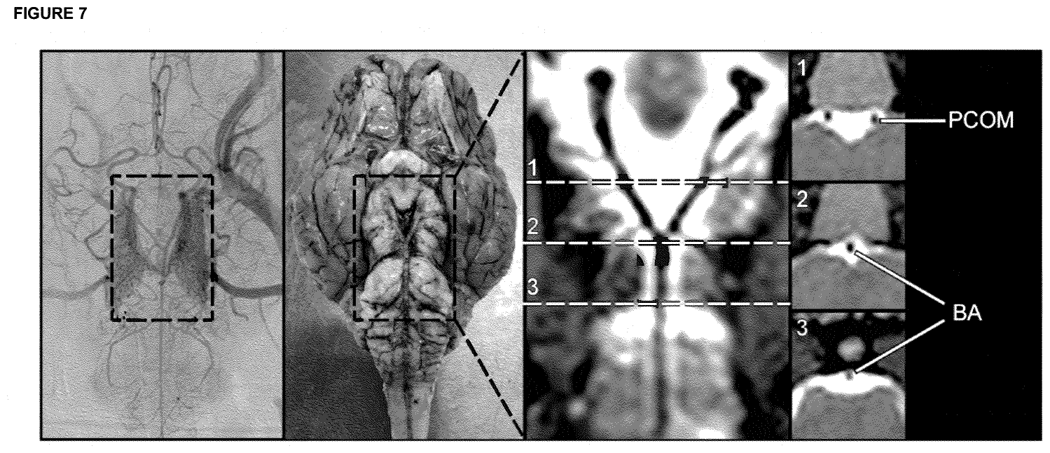

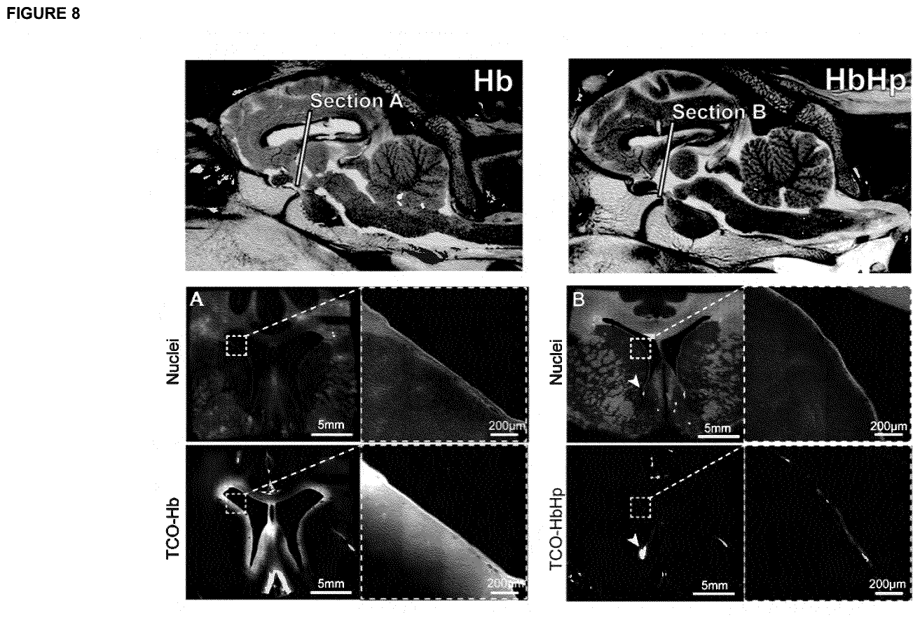

In the dynamic 3D MRI, MnHb and MnHb:Hp complexes injected into lateral ventricle was shown to rapidly distribute along the physiological internal CSF pathways through the third ventricle, aqueduct and fourth ventricle (n=5) (FIG. 7). Within minutes, a high signal of MnHb could be detected in the basal cisterns submerging the large cerebral arteries of the posterior fossa in all animals (FIG. 8). Further distribution to the middle and anterior cerebral fossa following the circle of Willis was observed within 20 minutes after injection. As expected, no difference between distribution of Hb and Hb:Hp complexes in the CSF compartment could be detected.

D. Haptoglobin Compartmentalizes Hb in the CSF Compartment and Reduces Penetration into Cerebral Vessel Walls and Brain Parenchyma

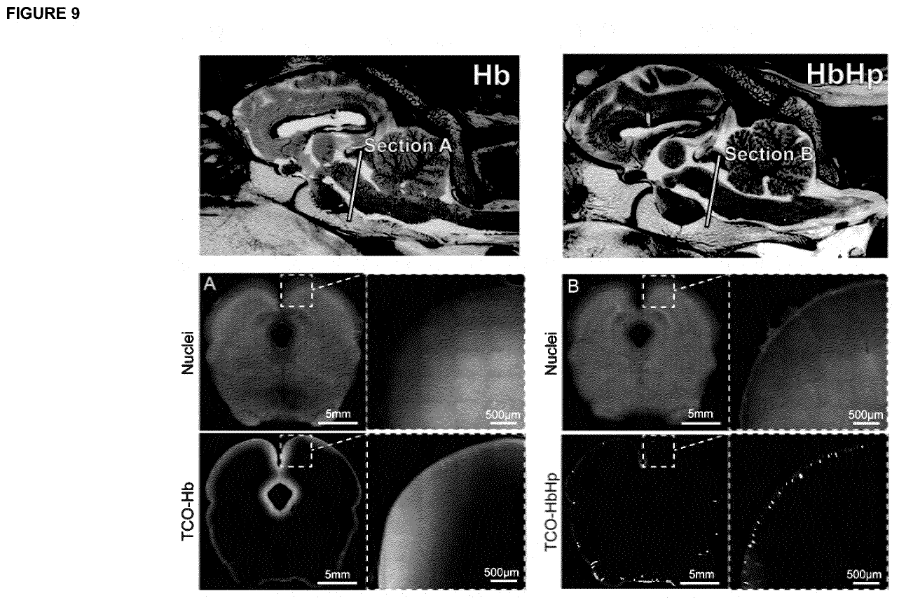

Histological analysis showed a penetration of TCO-labeled hemoglobin from CSF into brain interstitial space through the ependymal barrier of the ventricular system, whereas co-infusion of Hp drastically reduced this dissemination (FIG. 8). Furthermore, strong interstitial penetration of Hb was also seen from the subarachnoid space through the glia limitans of the brain surface (FIG. 9A). In contrast, Hb:Hp complexes were mainly restricted to the CSF-filled perivascular spaces (Virchow-Robin spaces) of penetrating cortical vessels (FIG. 9B).

In the systemic circulation size-restricted barriers prevent delocalization of the large Hb-Hp complex into vulnerable tissues such as the kidney, the myocardium or the vascular wall of resistance arteries. To translate this general concept of Hp protection to the brain, TCO-Hb or TCO-Hb-Hp complexes were infused into the CSF of sheep. The trans-cyclooctene(TCO)-tagged hemoproteins were visualized in formalin-fixed tissue sections by fluorescence microscopy at a very high signal-to-noise ratio after a click-chemistry reaction with a tetrazine-conjugated fluorochrome. The fluorescence scans of sheep brain sections at two different positions of the fore- and midbrain demonstrate that within 2 hours, the cell-free Hb delocalizes from the internal and external CSF spaces into the brain parenchyma, appearing as a rim of fluorescence-signal along the internal and external CSF-brain interfaces. This pattern of delocalization was absent in specimens of sheep that were infused with TCO-Hb-Hp complexes.

For the illustration of the Hb-Hp complex distribution in FIGS. 8 and 9, a section of the forebrain was chosen where the tip of the EVD catheter was placed slightly into the brain parenchyma. At this location, a small quantity of TCO-Hb-Hp was directly injected into the brain tissue serving as a positive control for the staining and imaging procedure. Besides this, the only distinct Hb-Hp signal could be recognized alongside small arteries penetrating from the pial surfaces into the brain. Before sacrificing the sheep we have collected CSF from the subarachnoid space for SEC and SDS-page analysis. Despite the absence of an Hb-Hp signal in brain sections, the large Hb-Hp complex were identified in the CSF composed of a ladder of type 2-2 Hp polymers that are intensely fluorescent after the TCO-reaction with Cy5-tetrazine. Collectively, these data support that the Hb-Hp complex remains compartmentalized in the CSF space. For confocal microscopy of vascular structures, sheep brain sections from animals infused with TCO-Hb, or TCO-Hb-Hp were stained for vascular smooth muscle cells (aSMA), and for the aquaporin-4 (AQP4) positive astrocyte end-feet.

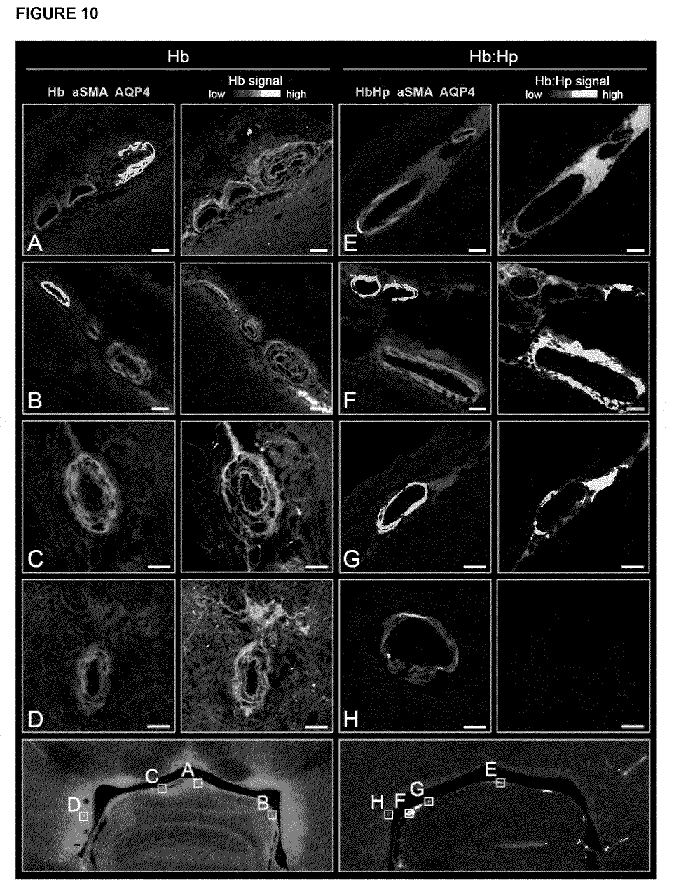

FIG. 10 shows confocal images of several small arteries of different calibers in the periventricular area of the midbrain of sheep that were infused with TCO-labeled Hb or Hb-Hp complexes, respectively. The images confirm that the Hb-Hp complex remains compartmentalized at a high concentration within the CSF-filled perivascular space (Virchow-Robin space) of penetrating arteries. This space is delineated by the outermost layer of the artery (i.e., adventitia) on one side and by the astrocyte end-feet on the other side (i.e., glia limitans). In the absence of Hp cell-free Hb delocalized across the astrocyte barrier into the brain tissue and additionally into the vascular smooth muscle layer reaching the subendothelial space. This observation may explain why cell-free Hb but not the large Hb-Hp complex interrupts vasodilatory NO signaling in cerebral arteries and thereby inducing vasospasm.

E. Cell-Free Hb in the Subarachnoid Space Induces Acute Vasospasms In Vivo which can be Prevented by Co-Infusion of Haptoglobin

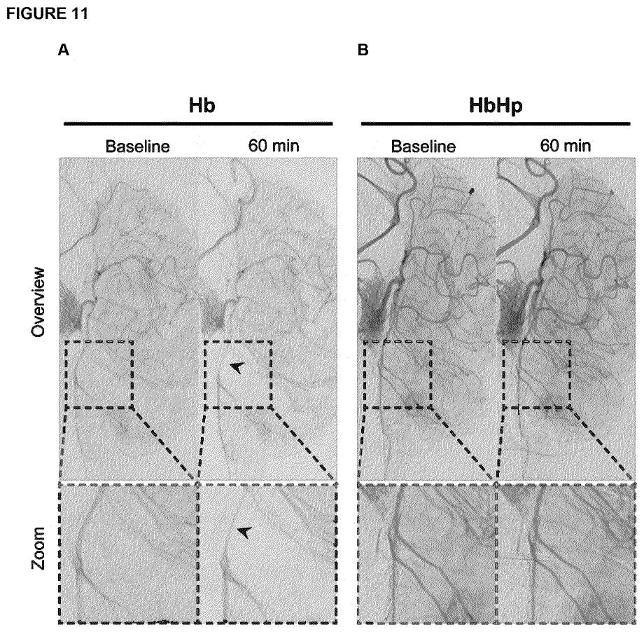

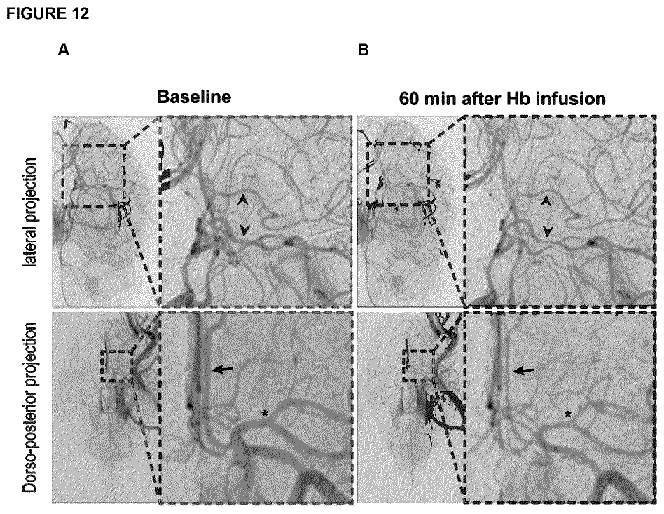

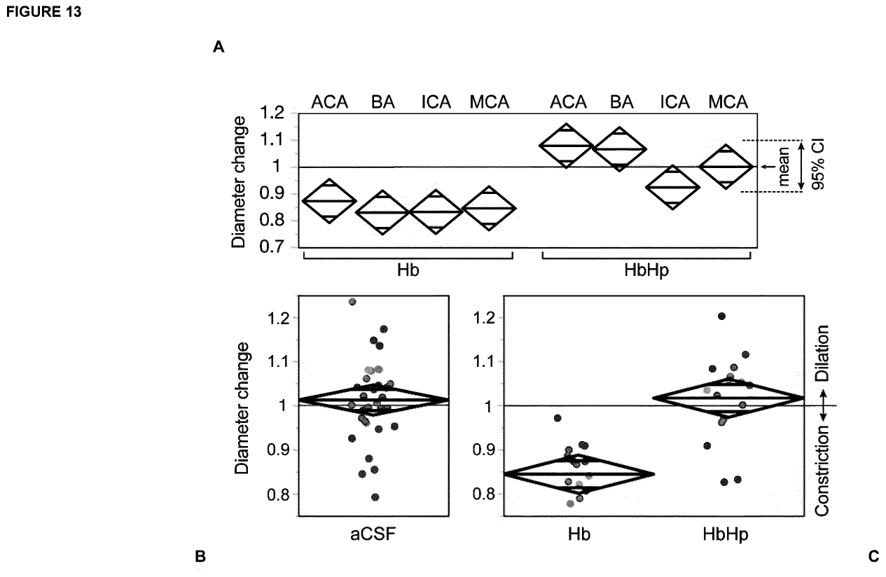

Cell-free Hb in the subarachnoid space induced acute angiographic vasospasms in 100% of animals (4/4). Vasospasms were reproducibly localized in the arteries of the posterior and anterior cranial fossa (basilar artery, cerebellar arteries, anterior cerebral artery) 60 minutes after ventricular injection of Hb (FIGS. 11A and 12B). However, vasospasms could also be detected in the medial cranial fossa (medial cerebral artery, internal carotid artery) with higher interindividual variations (FIG. 12). Co-infusion of Hp completely prevented induction of segmental arterial vasospasms (FIG. 11B). One animal with Hb:Hp co-infusion showed smaller vessel diameters in all analyzed vascular territories, without the segmental patterns of vasospasms seen in Hb-infused animals (data not shown). Semi-automated quantification of vessel diameter was performed on the DSA images of four ex-ante defined cerebral artery regions to objectify the visual impression of vasospasms. Significant narrowing of the basilar artery (BA) was observed in the anterior (ACA) and middle (MCA) cerebral arteries, as well as of the cisternal internal carotid artery (ICA) in Hb-infused compared to Hb:Hp infused animals (FIG. 13A). The Hb-induced vascular contraction became even more apparent in a pooled analysis of all arterial segments (FIG. 13B). Neither a significant change in arterial diameters after Hb-Hp infusion nor after infusion of an equal volume of artificial CSF was observed, which was performed as a general control procedure during the initial phase of each experiment (FIG. 13C).

F. Sequential Administration of Hp after Hb Infusion In Vivo Leads to Complete Complexation of Hb in CSF

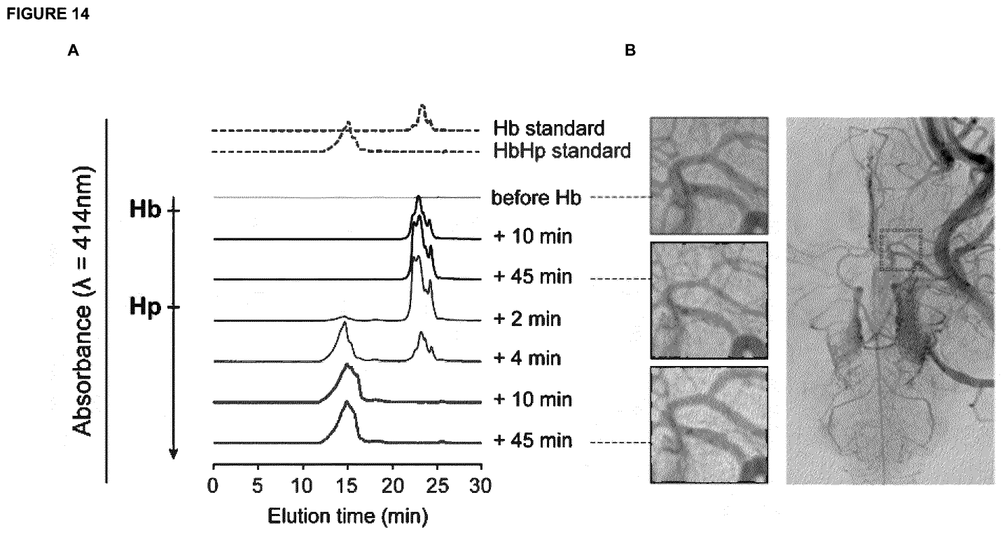

Sequentially injected Hp rapidly and completely binds free Hb dispersed in the subarachnoid space in vivo. Already 10 minutes after Hp administration to the ventricular system, no uncomplexed Hb was detectable in the suboccipitally collected CSF samples (FIG. 14).

The vasoconstrictive effect of sheep CSF collected at the time point of angiographic vasospasms in vivo can be neutralized by Hp in ex vivo vascular function experiments.

### G. The Vasoconstrictive Effect of Sheep CSF Collected at the Time Point of Angiographic Vasospasms In Vivo can be Neutralized by Hp in Ex Vivo Vascular Function Experiments

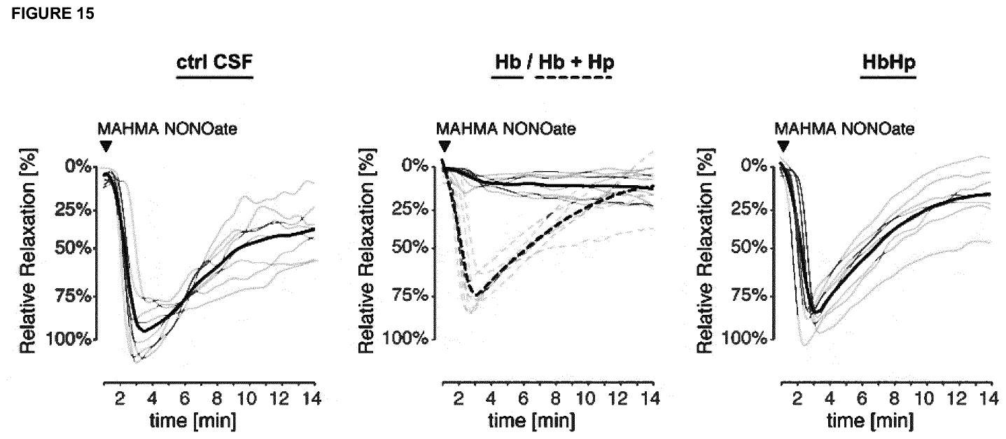

Isolated basilar arteries exposed to sheep CSF collected before Hb injection showed similar dilatory responses to NO like vessels in aCSF. After exposure to hemorrhagic CSF collected at the time point of angiographic vasospasms the NO response was massively reduced. Ex vivo treatment by addition of equimolar Hp (adjusted to individual Hb concentration in CSF samples) widely recovers the NO-responsiveness. Exposure to CSF collected 60 minutes after infusion of Hb:Hp complexes did not impair NO-induced vasodilation (FIG. 15).

H. Cell-Free Hb is the Major Disruptor of Arterial Nitric Oxide-Signaling in the Cerebrospinal Fluid of Patients with aSAH

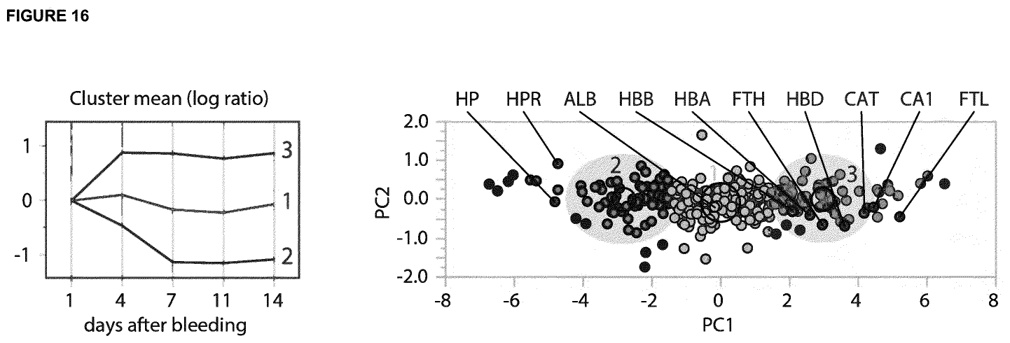

Quantitative LC-MSMS proteome analysis was performed on sequential CSF samples from patients with aSAH that were collected from the external ventricular drain (EVD) catheter between day 2 and 13 after bleeding. All proteins were classified that were identified with at least two unique peptides by a k-means clustering analysis of the log-transformed normalized ion intensity ratios (day x/day 2) (FIG. 16). Category 3 contained the proteins that remained unchanged over time. Category 2 encompassed proteins that increased over time. Category 2 was composed almost exclusively of red blood cell (RBC) components, namely Hb and RBC enzymes, illustrating the delayed erythrolytic process that occurs in the subarachnoid space after aSAH. Within Category 2, Hp was abundant in the initial samples but was found to be depleted over time, leaving an overwhelming fraction of cell-free Hb. Category 1 represents proteins that decreased over time and was primarily composed of plasma proteins.

I. Histomorphometric analysis of arterioles in the tela choroidea of the fourth

### Ventricle

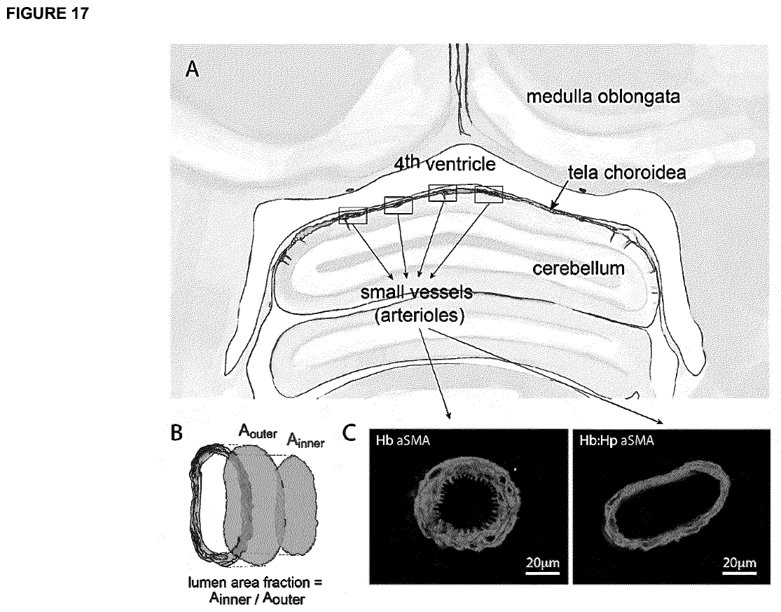

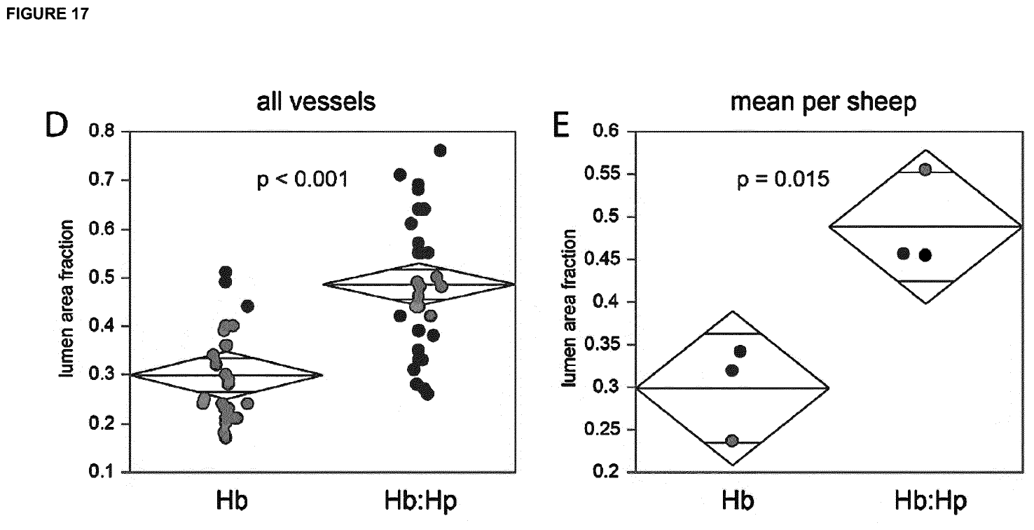

To link the observed perivascular Hb exposure of small parenchymal vessels (diameter range of 50-100 μm) to the functional readouts for large cerebral arteries in our studies, histomorphometric analysis was performed. For this purpose, 120 μm sheep brain sections through the fourth ventricle for smooth muscle cells were stained (FIG. 17A). Confocal images of the small alpha-smooth muscle actin (aSMA)-positive vessels in the tela choroidea were acquired by a researcher blinded to the treatments (n=3 sheep per group, a total of 25 images of Hb-treated sheep, 32 images for Hb-haptoglobin-treated sheep). The periventricular area was chosen for this analysis to ensure best possible structural preservation, because of its very rapid exposure to the intraventricularly instilled fixative. For each vessel, the lumen and total cross-sectional areas were quantified based on the inner and outer circumference of the aSMA positive structures, which were manually determined by three blinded researchers (FIG. 17B). While vessels with a contracted appearance were abundant in all Hb infused animals, none could be found in Hb-haptoglobin infused animals (FIG. 17C). FIGS. 17D and 17E show the quantitative analysis of the luminal fraction areas of all analysed vessels, as well as the mean per sheep. In both ways of analysis, the differences were statistically significant with smaller lumen areas of Hb compared to Hb-haptoglobin treated animals.

### SUMMARY AND CONCLUSION

In aSAH patients, delayed ischemic neurological deficits (DIND) occur mainly between posthemorrhagic day 4 and 14. This temporal profile correlates with the release of oxyHb from lysing erythrocytes into the subarachnoid compartment with CSF oxyHb peaks between day 4 and 14. In a small observational clinical study, a significant difference was observed between cumulative CSF oxyHb levels of aSAH patients with and without development of DIND. Secondary neurological deterioration is a major contributor to unfavourable outcome and prevention of DIND would be a game changer in the treatment of aSAH patients.

In this study, ex vivo and in vivo models were established to explore the effect of NO-scavenging through cell-free Hb on cerebral arteries and the potential of haptoglobin to prevent or otherwise reduce this toxicity. The concept of intramural NO-depletion and consecutive vasoconstriction through cell-free oxyHb was confirmed ex vivo in isolated porcine basilar arteries. Treatment of Hb-exposed arteries with haptoglobin fully restored NO-induced vasodilation. Further, the NO-dependent vasoactive effect of hemorrhagic CSF collected from aSAH patients was demonstrated in the same model. Despite numerous known vasoactive molecules in hemorrhagic CSF, scavenging of oxyHb by haptoglobin treatment restored the vasodilatory response to NO in isolated cerebral arteries to a physiological level. No difference in efficiency to restore the vasodilatory NO-response was observed between Hp 2-2 and Hp 1-1 (recombinant or plasma-derived).

To confirm the observed effects in vivo a highly translational large animal model was established. Injection of cell-free oxyHb into the CSF compartment in sheep induced reproducible angiographic vasospasms in all animals. More important from preventive perspectives, complexation of oxyHb with haptoglobin abolished this vasoreactivity in vivo. Additionally, the concept of therapeutic haptoglobin administration to restore NO-induced vasodilation was shown ex vivo in arteries exposed to hemorrhagic CSF collected from animals with angiographic vasospasms. These results strongly support the rationale for therapeutic haptoglobin administration in aSAH patients to prevent interference of cell-free oxyHb in CSF with cerebrovascular NO-signalling to reduce the incidence of adverse secondary neurological outcomes, including cerebral vasospasms and DIND.

To study the distribution of cell-free oxyHb injected into the CSF compartment, several novel labeling methods for in vivo and ex vivo imaging were developed. Using dynamic MRI, a rapid, macroscopically identical dispersion of Hb and Hb:Hp complexes in the subarachnoid space after injection into the ventricular system was confirmed. Histological analysis revealed an extensive penetration of Hb from CSF into the interstitial space of the brain, whereas Hb:Hp complexes was largely confined in the subarachnoid space. These unexpected findings strongly suggest that compartmentalization of cell-free Hb in the CSF spaces through complexation with haptoglobin contributes, at least in part, to the prevention of the toxic effects of cell-free Hb on cerebral vasculature and brain parenchyma, thereby highlighting the intrathecal administration of haptoglobin as therapeutic approach to reach sufficient Hb-scavenging capacity in the CSF compartment of aSAH patients.

The therapeutic potential of haptoglobin treatment may not be limited to aSAH patients, noting that all types of spontaneous or traumatic intracranial bleedings are accompanied by extravascular erythrolysis and release of cell-free Hb into the CSF and/or brain interstitial space. Therefore, a successful therapeutic approach to scavenge cell-free intracranial hemoglobin has an enormous potential impact on a large population of neurological patients.

### REFERENCES

1. Rincon F, Rossenwasser R H, Dumont A. The epidemiology of admissions of nontraumatic subarachnoid hemorrhage in the United States. Neurosurgery. 2013; 73:217-22; discussion 212-3.
2. Hughes J D, Bond K M, Mekary R A, Dewan M C, Rattani A, Baticulon R, et al. Estimating the Global Incidence of Aneurysmal Subarachnoid Hemorrhage: A Systematic Review for Central Nervous System Vascular Lesions and Meta-Analysis of Ruptured Aneurysms. World Neurosurg. 2018; 115:430-447.e7.
3. Nieuwkamp D J, Setz L E, Algra A, Linn F H H, de Rooij N K, Rinkel G J E. Changes in case fatality of aneurysmal subarachnoid haemorrhage over time, according to age, sex, and region: a meta-analysis. Lancet Neurol. 2009; 8:635-642.
4. Taufique Z, May T, Meyers E, Falo C, Mayer S A, Agarwal S, et al. Predictors of Poor Quality of Life 1 Year After Subarachnoid Hemorrhage. Neurosurgery. 2016; 78:256-264.
5. Buchanan K M, Elias L J, Goplen G B. Differing perspectives on outcome after subarachnoid hemorrhage: the patient, the relative, the neurosurgeon. Neurosurgery. 2000; 46:831-8; discussion 838-40.
6. Morris P G, Wilson J T L, Dunn L. Anxiety and depression after spontaneous subarachnoid hemorrhage. Neurosurgery. 2004; 54:47-52; discussion 52-4.
7. Loch Macdonald R. Delayed neurological deterioration after subarachnoid haemorrhage. Nat. Rev. Neurol. 2013; 10:44-58.
8. Pluta R M, Afshar J K, Boock R J, Oldfield E H. Temporal changes in perivascular concentrations of oxyhemoglobin, deoxyhemoglobin, and methemoglobin after subarachnoid hemorrhage. J. Neurosurg. 1998; 88:557-561.
9. Willenborg D O, Staykova M, Fordham S, O'Brien N, Linares D. The contribution of nitric oxide and interferon gamma to the regulation of the neuro-inflammation in experimental autoimmune encephalomyelitis. J. Neuroimmunol. 2007; 191:16-25.
10. Guzik T J, Korbut R, Adamek-Guzik T. Nitric oxide and superoxide in inflammation and immune regulation. J. Physiol. Pharmacol. 2003; 54:469-487.
11. Lawson C L, Hanson R J. Solving least squares problems. SIAM; 1995.
12. Lipiski M, Deuel J W, Baek J H, Engelsberger W R, Buehler P W, Schaer D J. Human Hp1-1 and Hp2-2 phenotype-specific haptoglobin therapeutics are both effective in vitro and in guinea pigs to attenuate hemoglobin toxicity. Antioxid. Redox Signal. 2013; 19:1619-1633.
13. Fischer M J M, Uchida S, Messlinger K. Measurement of meningeal blood vessel diameter in vivo with a plug-in for ImageJ. Microvasc. Res. 2010; 80:258-266.
14. Elmer J, Harris D R, Sun G, Palmer A F. Purification of hemoglobin by tangential flow filtration with diafiltration. Biotechnol. Prog. 2009; 25:1402-1410.
15. Dumont A S, Dumont R J, Chow M M, Lin C L, Calisaneller T, Ley K F, Kassell N F, Lee K S. Chapter Two: Cerebral vasospasm after subarachnoid hemorrhage: putative role of inflammation. Neurosurgery 2003.

1. A method of treating or preventing an adverse secondary neurological outcome in a subject following a haemorrhagic stroke accompanied by extravascular erythrolysis and release of cell-free haemoglobin (Hb) into a cerebral spinal fluid (CSF), the method comprising exposing the CSF of a subject in need thereof to a therapeutically effective amount of haptoglobin (Hp) and for a period of time sufficient to allow the Hp to form a complex with, and thereby neutralise, the cell-free Hb.

2. The method of claim 1, wherein the haemorrhagic stroke is a spontaneous haemorrhage, a traumatic haemorrhage, an intraventricular haemorrhage, or a subarachnoid haemorrhage.

3. (canceled)

4. The method of claim 2, wherein the subarachnoid haemorrhage is an aneurysmal subarachnoid haemorrhage.

5. The method of claim 1, wherein the adverse secondary neurological outcome is selected from the group consisting of a delayed ischaemic neurological deficit (DIND), delayed cerebral ischaemia (DCI), neurotoxicity, inflammation, nitric oxide depletion, oxidative tissue injury, cerebral vasospasm, cerebral vasoreactivity, within the brain parenchyma and oedema.

6. (canceled)

7. (canceled)

8. (canceled)

9. (canceled)

10. The method of claim 1, comprising exposing the CSF to the Hp, or the functional analogue thereof, within 21 days after onset of the haemorrhage.

11. The method of claim 10, comprising exposing the CSF to the Hp from 2 days to 4 days after onset of the haemorrhage.

12. The method of claim 10, comprising exposing the CSF to the Hp from 5 days to 14 days after onset of the haemorrhage.

13. The method of claim 1, comprising intracranially, intrathecally, or intracerebroventricularly administering to the subject the therapeutically effective amount of the Hp.

14. (canceled)

15. The method of claim 13, wherein the Hp is intrathecally administering into the spinal canal or into the subarachnoid space.

16. (canceled)

17. (canceled)

18. The method of claim 13, wherein the period of time to which the CSF is exposed to the therapeutically effective amount of Hp is at least 2 minutes.

19. (canceled)

20. (canceled)

21. (canceled)

22. The method of claim 1, wherein the therapeutically effective amount of Hp is at least an equimolar amount to the concentration of cell-free Hb in the CSF of the subject following the haemorrhage.

23. The method of claim 22, further comprising (i) obtaining a CSF sample from the subject following the haemorrhage and prior to exposing the CSF to the Hp; (ii) measuring the amount of cell-free Hb in the CSF sample obtained in step (i); and (iii) determining the at least equimolar amount of Hp based on the concentration of cell-free Hb from step (ii).

24. The method of claim 1, wherein the therapeutically effective amount of Hp is from 2 μM to 20 mM.

25. (canceled)

26. (canceled)

27. (canceled)

28. The method of claim 1, further comprising removing Hp:cell-free Hb complexes formed in the CSF.

29. The method of claim 1, comprising exposing the CSF to the therapeutically effective amount of Hp extracorporeally.

30. The method of claim 29, comprising:
(i) obtaining sample of CSF from the CSF compartment of the subject following the haemorrhage;
(ii) adding to the CSF sample of step (i) Hp to obtain an Hp-enriched CSF sample;
(iii) administering the Hp-enriched CSF sample to the subject, thereby exposing the CSF compartment of the subject to a therapeutically effective amount of Hp for a period of time sufficient to allow the Hp to form a complex with cell-free Hb in the CSF compartment of the subject; and
(iv) optionally repeating steps (i) to (iii).

31. The method of claim 29, comprising:
(i) removing a volume of CSF from the CSF compartment of the subject following the haemorrhage;
(ii) providing an artificial CSF comprising Hp;
(iii) administering the artificial CSF of (ii) to the subject, thereby exposing the CSF compartment of the subject to a therapeutically effective amount of Hp for a period of time sufficient to allow the Hp to form a complex with cell-free Hb in the CSF compartment of the subject; and
(iv) optionally repeating steps (i) to (iii).

32. The method of claim 29, comprising:
(i) obtaining CSF from the subject following the haemorrhage;
(ii) exposing the CSF from step (i) to the Hp under conditions to allow the Hp, or the functional analogue thereof, to form a complex with cell-free Hb in the CSF;
(iii) extracting the Hp:cell-free Hb complexes from the CSF following step (ii) to obtain an Hb-diminished CSF that has a lower amount of cell-free Hb when compared to the CSF from step (i);
(iv) optionally repeating steps (ii) and (iii) to obtain an Hb-diminished CSF that is substantially free of cell-free Hb; and
(v) administering the Hb-diminished CSF obtained from step (iii) or step (iv) to the CSF compartment of the subject.

33. The method of claim 32, further comprising adding to the Hb-diminished CSF prior to step (vi) a therapeutically effective amount of Hp.

34. The method of claim 32, wherein the Hp is immobilised on a substrate.

35. The method of claim 34, wherein the substrate is an affinity chromatography resin.

36. The method of claim 35, wherein step (ii) comprises passing the CSF from step (i) through an affinity chromatography resin under conditions that allow the cell-free Hb in the CSF to bind to the resin; wherein step (iii) comprises eluting the CSF from the resin following step (ii); and wherein step (iv) comprising recovering the eluted CSF.

37. The method of claim 32, further comprising washing the CSF compartment following step (i) with a wash solution.

38. The method of claim 37, wherein the wash solution is an artificial CSF.

39. The method of claim 31, wherein the artificial CSF comprises NaCl, KCl, KH₂PO₄, NaHCO₃, MgCl₆H₂O, CaCl₂H₂O and glucose.

40. The method of claim 37, wherein the wash solution comprises Hp.

41. The method of claim 40, wherein the wash solution comprises from 2 μM to 20 mM Hp.

42. (canceled)

43. The method of claim 1, comprising:
(i) removing the CSF from the subject following the haemorrhage;
(ii) rinsing the CSF compartment of the subject following step (i) with a wash solution comprising a therapeutically effective amount of Hp;
(iii) optionally repeating step (ii); and
(iv) administering to the CSF compartment of the subject, following step (ii) or step (iii), an artificial CSF.

44. The method of claim 43, wherein the artificial CSF comprises NaCl, KCl, KH₂PO₄, NaHCO₃, MgCl₆H₂O, CaCl₂)H₂O and glucose.

45. The method of claim 43, wherein the artificial CSF comprises Hp.

46. The method of claim 45, wherein the artificial CSF comprises from 2 μM to 20 mM Hp.

47. (canceled)

48. The method of claim 1, wherein the Hp is selected from the group consisting of an Hp1-1 homodimer, an Hp1-2 multimer, an Hp2-2 multimer, and a combination of any of the foregoing.

49. (canceled)

50. The method of claim 48, wherein the Hp is a recombinant protein.

51. The method of claim 50, wherein the Hp is plasma derived.

52. The method of claim 51, further comprising administering to the subject a second agent for treating or preventing an adverse secondary neurological outcome following an intraventricular haemorrhage.

53. The method of claim 52, wherein the second agent is a vasodilator.

54. The method of claim 53, wherein the second agent is selected from the group consisting of a sydnone and sodium nitroprusside.

55. An artificial cerebral spinal fluid (CSF) comprising Hp.

56. (canceled)

57. (canceled)

58. (canceled)

59. (canceled)

60. (canceled)

61. (canceled)

62. (canceled)

63. (canceled)

64. A pharmaceutical composition comprising a therapeutically effective amount of Hp and a pharmaceutically acceptable carrier.

65.-74. (canceled)
